SEQUENCE LISTING (127 sequences)

SEQ ID NO: 1-9 - skipped (9 entries)

SEQ ID NO: 10 - protein, 15 aa, synthetic construct
  REGION 1..15: note = Synthetic Construct
RASKGVSTSGYSYLH

SEQ ID NO: 11 - protein, 7 aa, synthetic construct
  REGION 1..7: note = Synthetic Construct
LASYLES

SEQ ID NO: 12 - protein, 9 aa, synthetic construct
  REGION 1..9: note = Synthetic Construct
QHSRDLPLT

SEQ ID NO: 13 - protein, 111 aa, synthetic construct [sequence omitted]

SEQ ID NO: 14 - protein, 218 aa, synthetic construct
  REGION 1..218: note = Synthetic Construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 5 aa, synthetic construct
  REGION 1..5: note = Synthetic Construct
NYYMY

SEQ ID NO: 16 - protein, 17 aa, synthetic construct
  REGION 1..17: note = Synthetic Construct
GINPSNGGTNFNEKFKN

SEQ ID NO: 17 - protein, 11 aa, synthetic construct
  REGION 1..11: note = Synthetic Construct
RDYRFDMGFDY

SEQ ID NO: 18 - protein, 120 aa, synthetic construct [sequence omitted]

SEQ ID NO: 19 - protein, 447 aa, synthetic construct
  REGION 1..447: note = Synthetic Construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20-22 - skipped (3 entries)

SEQ ID NO: 23 - protein, 12 aa, synthetic construct
  REGION 1..12: note = Synthetic Construct
YSISSGYHWSWI

SEQ ID NO: 24-34 - skipped (11 entries)

SEQ ID NO: 35 - protein, 20 aa, synthetic construct
  REGION 1..20: note = Synthetic Construct
LARIDWDDDKYYSTSLKSRL

SEQ ID NO: 36-44 - skipped (9 entries)

SEQ ID NO: 45 - protein, 9 aa, synthetic construct
  REGION 1..9: note = Synthetic Construct
ARSYVYFDY

SEQ ID NO: 46-57 - skipped (12 entries)

SEQ ID NO: 58 - protein, 12 aa, synthetic construct
  REGION 1..12: note = Synthetic Construct
RASQSVRGRFLA

SEQ ID NO: 59-65 - skipped (7 entries)

SEQ ID NO: 66 - protein, 9 aa, synthetic construct
  REGION 1..9: note = Synthetic Construct
DASNRATGI

SEQ ID NO: 67-74 - skipped (8 entries)

SEQ ID NO: 75 - protein, 11 aa, synthetic construct
  REGION 1..11: note = Synthetic Construct
YCQQSSSWPPT

SEQ ID NO: 76-86 - skipped (11 entries)

SEQ ID NO: 87 - protein, 116 aa, synthetic construct
  REGION 1..116: note = Synthetic Construct
EVQLVESGGGLVQPGGSLRLSCAASGYSISSGYHWSWIRQAPGKGLEWLARIDWDDDKYYSTSLKSRLTISRDNSKNTLYLQLNSLRAEDTAVYYCARSYVYFDYWGQGTLVTVSS

SEQ ID NO: 88-99 - skipped (12 entries)

SEQ ID NO: 100 - protein, 109 aa, synthetic construct
  REGION 1..109: note = Synthetic Construct
DIQLTQSPSSLSASVGDRVTITCRASQSVRGRFLAWYQQKPGKAPKLLIYDASNRATGIPSRFSGSGSGTDFTLTISSLQPEDFATYYCQQSSSWPPTFGQGTKVEIKR

SEQ ID NO: 101-124 - skipped (24 entries)

SEQ ID NO: 125 - protein, 445 aa, synthetic construct
  REGION 1..445: note = Synthetic Construct
EVQLVESGGGLVQPGGSLRLSCAASGYSISSGYHWSWIRQAPGKGLEWLARIDWDDDKYYSTSLKSRLTISRDNSKNTLYLQLNSLRAEDTAVYYCARSYVYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 126 - protein, 446 aa, synthetic construct
  REGION 1..446: note = Synthetic Construct
EVQLVESGGGLVQPGGSLRLSCAASGYSISSGYHWSWIRQAPGKGLEWLARIDWDDDKYYSTSLKSRLTISRDNSKNTLYLQLNSLRAEDTAVYYCARSYVYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 215 aa, synthetic construct
  REGION 1..215: note = Synthetic Construct
DIQLTQSPSSLSASVGDRVTITCRASQSVRGRFLAWYQQKPGKAPKLLIYDASNRATGIPSRFSGSGSGTDFTLTISSLQPEDFATYYCQQSSSWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0166142 A1
(43) Pub. Date: Jun. 18, 2026

(54) METHODS FOR TREATING CANCER USING ANTI-CTLA4 ANTIBODY IN COMBINATION WITH PEMBROLIZUMAB

(71) Applicant: ADAGENE PTE. LTD., Singapore (SG)

(72) Inventors: Peter Peizhi LUO, Morgan Hill, CA (US); Jiping ZHA, Suzhou, Jiangsu (CN); Songmao ZHENG, West Harrison, IN (US); Guizhong LIU, Fort Lee, NJ (US); Xiaohong SHE, Suzhou, Jiangsu (CN)

(73) Assignee: ADAGENE PTE. LTD., Singapore (SG)

(21) Appl. No.: 19/127,023

(22) PCT Filed: Nov. 2, 2023
(86) PCT No.: PCT/US2023/078546

§ 371 Date: May 2, 2025

Related U.S. Application Data
(60) Provisional application No. 63/422,947, filed on Nov. 5, 2022.

(51) Int. Cl. A61K 39/395; A61K 39/00; A61P 35/00; C07K 16/28

(52) U.S. Cl. CPC A61K 39/3955; A61P 35/00; C07K 16/2818; A61K 2039/507; A61K 2039/545; C07K 2317/24

(57) ABSTRACT

The present application provides compositions and methods for treating cancers, including advanced-stage metastatic cancer, using an anti-CTLA4 antibody in combination with pembrolizumab.

### CROSS-REFERENCE TO RELATED APPLICATIONS

This application claims priority to U.S. Provisional Patent Application No. 63/422,947 filed Nov. 5, 2022, the disclosure of which is hereby incorporated by reference in its entirety for all purposes.

### REFERENCE TO AN ELECTRONIC SEQUENCE LISTING

The contents of the electronic sequence listing (695402002640seqlist.xml; Size: 50,377 bytes; and Date of Creation: Nov. 1, 2023) is herein incorporated by reference in its entirety.

### FIELD OF THE INVENTION

The present application is in the field of cancer therapeutics, and relates to compositions and methods for treating cancers using an antibody that binds to human CTLA4 in combination with the anti-PD-1 antibody pembrolizumab.

### BACKGROUND

CTLA4 is a member of the immunoglobulin (Ig) superfamily of proteins that acts to downregulate T-cell activation and maintain immunogenic homeostasis. It has been shown that in vivo antibody-mediated blockade of CTLA4 enhanced anti-cancer immune responses in a syngeneic murine prostate cancer model (K won et al. (1997) Proc Natl Acad Sci USA, 94 (15): 8099-103). In addition, blockade of CTLA4 function was shown to enhance anti-tumor T cell responses at various stages of tumor growth in tumor-bearing mice (Yang et al. (1997) Cancer Res 57 (18): 4036-41; Hurwitz et al. (1998) Proc Natl Acad Sci USA 95 (17): 10067-7). However, the development of antibody-based therapeutics suitable for human use remains difficult, as translation from pre-clinical animal models to human safety is often poor. Accordingly, a need exists for anti-CTLA4 antibodies that are cross-reactive among different species, such as humans and experimental animals (e.g., mouse, monkey, rat, etc.), to concurrently enable animal model studies and provide suitable human therapeutic candidates. In addition, a need exists for the development of safer anti-CTLA4 antibodies that are only active in certain contexts, such as in the protease-rich tumor microenvironment.

PD-1 is recognized as an important molecule in immune regulation and the maintenance of peripheral tolerance. PD-1 is moderately expressed on naive T, B and NKT cells and up-regulated by T/B cell receptor signaling on lymphocytes, monocytes and myeloid cells (Sharpe, Arlene H et al., The function of programmed cell death 1 and its ligands in regulating autoimmunity and infection. Nature Immunology (2007); 8:239-245).

Two known ligands for PD-1, PD-L1 (B7-H1) and PD-L2 (B7-DC), are expressed in human cancers arising in various tissues. In large sample sets of e.g. ovarian, renal, colorectal, pancreatic, liver cancers and melanoma, it was shown that PD-L1 expression correlated with poor prognosis and reduced overall survival irrespective of subsequent treatment (Dong, Haidong et al., Tumor-associated B7-H1 promotes T-cell apoptosis: a potential mechanism of immune evasion. Nat Med. 2002 August; 8(8): 793-800; Yang, Wanhua et al., PD-1 interaction contributes to the functional suppression of T-cell responses to human uveal melanoma cells in vitro. Invest Ophthalmol Vis Sci. 2008 June; 49 (6 (2008): 49: 2518-2525; Ghebeh, Hazem et al., The B7-H1 (PD-L1) T lymphocyte-inhibitory molecule is expressed in breast cancer patients with infiltrating ductal carcinoma: correlation with important high-risk prognostic factors. Neoplasia (2006) 8:190-198; Hamanishi, Junzo et al., Programmed cell death 1 ligand 1 and tumor-infiltrating CD8+ T lymphocytes are prognostic factors of human ovarian cancer. Proc. Natl. Acad. Sci. USA (2007): 104: 3360-3365; Thompson, R Houston, and Eugene D Kwon, Significance of B7-H1 overexpression in kidney cancer. Clinical genitourin Cancer (2006): 5: 206-211; Nomi, Takeo et al., Clinical significance and therapeutic potential of the programmed death-1 ligand/programmed death-1 pathway in human pancreatic cancer. Clinical Cancer Research (2007); 13:2151-2157; Ohigashi, Yuichiro et al., Clinical significance of programmed death-1 ligand-1 and programmed death-1 ligand 2 expression in human esophageal cancer. Clin. Cancer Research (2005): 11: 2947-2953; Inman, Brant A et al., PD-L1 (B7-H1) expression by urothelial carcinoma of the bladder and BCG-induced granulomata: associations with localized stage progression. Cancer (2007): 109: 1499-1505; Shimauchi, Takatoshi et al., Augmented expression of programmed death-1 in both neoplasmatic and nonneoplastic CD4+ T-cells in adult T-cell Leukemia/Lymphoma. Int. J. Cancer (2007): 121:2585-2590; Gao, Qiang et al., Overexpression of PD-L1 significantly associates with tumor aggressiveness and postoperative recurrence in human hepatocellular carcinoma. Clinical Cancer Research (2009) 15:971-979; Nakanishi, Juro et al., Overexpression of B7-H1 (PD-L1) significantly associates with tumor grade and postoperative prognosis in human urothelial cancers. Cancer Immunol Immunother. (2007) 56:1173-1182; Hino et al., Tumor cell expression of programmed cell death-1 is a prognostic factor for malignant melanoma. Cancer (2010): 00: 1-9). Similarly, PD-1 expression on tumor infiltrating lymphocytes was found to mark dysfunctional T cells in breast cancer and melanoma (Ghebeh, Hazem et al., Foxp3+ tregs and B7-H1+/PD-1+ T lymphocytes co-infiltrate the tumor tissues of high-risk breast cancer patients: implication for immunotherapy. BMC Cancer. 2008 Feb. 23; 8:57; Ahmadzadeh, Mojgan et al., Tumor antigen-specific CD8 T cells infiltrating the tumor express high levels of PD-1 and are functionally impaired. Blood (2009) 114:1537-1544) and to correlate with poor prognosis in renal cancer (Thompson, R Houston et al., PD-1 is expressed by tumor infiltrating cells and is associated with poor outcome for patients with renal carcinoma. Clinical Cancer Research (2007) 15:1757-1761). Thus, it has been proposed that PD-L1-expressing tumor cells interact with PD-1-expressing T cells to attenuate T cell activation and evasion of immune surveillance, thereby contributing to an impaired immune response against the tumor.

Several monoclonal antibodies that inhibit the interaction between PD-1 and one or both of its ligands PD-L1 and PD-L2 have been approved for treating cancer. Pembrolizumab (KEYTRUDA®, Merck Sharp & Dohme LLC, Rahway, NJ, USA) is a potent humanized immunoglobulin G4 (IgG4) mAb with high specificity of binding to the programmed cell death 1 (PD-1) receptor, thus inhibiting its interaction with programmed cell death ligand 1 (PD-L1) and programmed cell death ligand 2 (PD-L2). Based on preclinical in vitro data, pembrolizumab has high affinity and potent receptor blocking activity for PD-1. Keytruda® (pembrolizumab) is indicated for the treatment of patients across a number of indications and is indicated for the first-line treatment of patients with unresectable or metastatic CRC that is microsatellite instability-high or mismatch repair deficient (MSI-H/dMMR). Pembrolizumab is the current standard of care for first line MSI-H/dMMR mCRC.

### BRIEF SUMMARY

The present application provides methods of treating cancer with an anti-CTLA4 antibody of the disclosure in combination with the anti-PD-1 antibody pembrolizumab. The anti-CTLA4 antibody comprises a heavy chain variable region and a light chain variable region, wherein the heavy chain variable region comprises an HVR-H1, an HVR-H2, and an HVR-H3, and the light chain variable region comprises an HVR-L1, an HVR-L2, and an HVR-L3, wherein the HVR-H1 comprises an amino acid sequence according to a formula YSISSGYHWSWI (SEQ ID NO: 23), the HVR-H2 comprises an amino acid sequence according to a formula LARIDWDDDKYYSTSLKSRL (SEQ ID NO: 35), the HVR-H3 comprises an amino acid sequence according to a formula ARSYVYFDY (SEQ ID NO: 45), the HVR-L1 comprises an amino acid sequence according to a formula RASQSVRGRFLA (SEQ ID NO: 58), the HVR-L2 comprises an amino acid sequence according to a formula DASNRATGI (SEQ ID NO: 66), and the HVR-L3 comprises an amino acid sequence according to a formula YCQQSSSWPPT (SEQ ID NO: 75).

In some embodiments, the anti-CTLA4 antibody comprises a heavy chain variable region comprising the amino acid sequence of SEQ ID NO: 87 or a variant thereof having at least about 90% (e.g., at least about 92%, 95%, 98%, 99% or more) sequence identity to the amino acid sequence of SEQ ID NO: 87, and a light chain variable region comprising the amino acid sequence of SEQ ID NO: 100 or a variant thereof having at least about 90% (e.g., at least about 92%, 95%, 98%, 99% or more) sequence identity to the amino acid sequence of SEQ ID NO: 100. In some embodiments, the anti-CTLA4 antibody comprises a heavy chain variable region comprising the amino acid sequence of SEQ ID NO: 87 and light chain variable region comprising the amino acid sequence of SEQ ID NO: 100.

In some embodiments, the anti-CTLA4 antibody comprises a heavy chain region comprising the acid sequence of amino EVOLVESGGGLVQPGGSLRLSCAASGYSISSGYHWSWIRQAPGKGLEWLARIDWDDDKYYSTSL KSRLTISRDNSKNTLYLOLNSLRAEDTAVYYCARSYVYFDYWGQGTLVTVSSASTKGPSVFPLA PSSKSTSGGTAALGCLVKDYFPEPVTVSWNSGALTSGVHTFPAVLQSSGLYSLSSVVTVPSSSL GTQTYICNVNHKPSNTKVDKKVEPKSCDKTHTCPPCPAPELLGGPSVFLFPPKPKDTLMISRTP EVTCVVVDVSHEDPEVKFNWYVDGVEVHNAKTKPREEQYNSTYRVVSVLTVLHQDWLNGKEYKC KVSNKALPAPIEKTISKAKGQPREPQVYTLPPSRDELTKNQVSLTCLVKGFYPSDIAVEWESNG QPENNYKTTPPVLDSDGSFFLYSKLTVDKSRWQQGNVFSCSVMHEALHNHYTOKSLSLSPGK (SEQ ID NO:126) or a variant thereof having at least about 90% (e.g., at least about 92%, 95%, 98%, 99% or more) sequence identity to the amino acid sequence of SEQ ID NO: 126, and a light chain region comprising the amino acid sequence of DIQLTQSPSSLSASVGDRVTITCRASQSVRGRFLAWYQQKPGKAPKLLIYDASNRATGIPSRFS GSGSGTDFTLTISSLOPEDFATYYCOQSSSWPPTFGQGTKVEIKRTVAAPSVFIFPPSDEQLKS GTASVVCLLNNFYPREAKVQWKVDNALQSGNSQESVTEQDSKDSTYSLSSTLTLSKADYEKHKV YACEVTHQGLSSPVTKSENRGEC (SEQ ID NO:127) or a variant thereof having at least about 90% (e.g., at least about 92%, 95%, 98%, 99% or more) sequence identity to the amino acid sequence of SEQ ID NO: 127. In some embodiments, the anti-CTLA4 antibody is TY21580, which comprises a heavy chain region comprising the amino acid sequence of SEQ ID NO: 126 and light chain region comprising the amino acid sequence of SEQ ID NO: 127.

In one aspect, the disclosure provides a method of treating a cancer in a subject, comprising administering to the subject an effective amount of an anti-CTLA4 antibody described above (e.g., TY21580) in combination with pembrolizumab, wherein the anti-CTLA4 antibody is administered at a dose of from about 3 mg/kg to about 10 mg/kg. In some embodiments, the anti-CTLA4 antibody (e.g., TY21580) is administered at a dose of about 3 mg/kg. In some embodiments, the anti-CTLA4 antibody (e.g., TY21580) is administered at a dose of about 5 mg/kg. In some embodiments, the anti-CTLA4 antibody (e.g., TY21580) is administered at a dose of about 6 mg/kg. In some embodiments, the anti-CTLA4 antibody (e.g., TY21580) is administered at a dose of about 8 mg/kg. In some embodiments, the anti-CTLA4 antibody (e.g., TY21580) is administered at a dose of about 10 mg/kg.

In one embodiment, the anti-CTLA4 antibody (e.g., TY21580) is administered to the subject at dose of from about 1 mg/kg to about 10 mg/kg or from about 2 mg/kg to about 5 mg/kg. In some such embodiments, the anti-CTLA4 antibody (e.g., TY21580) is administered to the subject at a dose of about 3 mg/kg. In some embodiments, the anti-CTLA4 antibody (e.g., TY21580) is administered to the subject once every three weeks. In other embodiments, the anti-CTLA4 antibody (e.g., TY21580) is administered to the subject once every six weeks. In particular embodiments, the anti-CTLA4 antibody (e.g., TY21580) is administered to a patient at a dose of about 3 mg/kg once every three weeks or once every six weeks. In any of the foregoing embodiments, pembrolizumab can be administered in combination with the anti-CTLA4 antibody on the same day of a particular dosing regimen or on different days of a particular regimen. In some embodiments, both the anti-CTLA4 antibody and pembrolizumab are administered on the first day of a three week or six week dosing regimen.

In some embodiments, the pembrolizumab is administered at a dose of from about 100 mg to about 300 mg once every three weeks. In some embodiments, the pembrolizumab is administered at a dose of about 200 mg once every three weeks. In some such embodiments, the anti-CTLA4 antibody is administered concurrently with the pembrolizumab. For instance, the anti-CTLA4 antibody and the pembrolizumab can each be administered to a patient in need thereof on day 1 of a three week or a six week dosing schedule.

In some embodiments, the anti-CTLA4 antibody and pembrolizumab are administered to a patient in need thereof once every three weeks. In some such embodiments, the anti-CTLA4 antibody is administered at a dose of from about 2 mg/kg to about 5 mg/kg (e.g., 3 mg/kg) and the pembrolizumab is administered at a dose of from about 100 mg/kg to about 300 mg/kg (e.g., about 200 mg). In particular embodiments, the anti-CTLA4 antibody and pembrolizumab are administered concurrently.

In some embodiments, the pembrolizumab is administered at a dose of from about 200 mg to about 400 mg once every six weeks. In some embodiments, the pembrolizumab is administered at a dose of about 400 mg once every six weeks. In some such embodiments, the anti-CTLA4 antibody is administered concurrently with the pembrolizumab. For instance, the anti-CTLA4 antibody and the pembrolizumab can each be administered to a patient in need thereof on day 1 of a three week or a six week dosing schedule.

In some embodiments, the anti-CTLA4 antibody and pembrolizumab are administered to a patient in need thereof once every six weeks. In some such embodiments, the anti-CTLA4 antibody is administered at a dose of from about 2 mg/kg to about 5 mg/kg (e.g., 3 mg/kg) and the pembrolizumab is administered at a dose of from about 200 mg to about 400 mg (e.g., about 400 mg). In particular embodiments, the anti-CTLA4 antibody and pembrolizumab are administered concurrently.

In some embodiments according to any one of the methods described above, the cancer is resistant or refractory to a prior therapy, wherein the prior therapy is an inhibitor of CTLA4, PD-1, or a PD-1 ligand. In some embodiments, the subject is resistant to or has relapsed from a prior therapy, wherein the prior therapy is an inhibitor of CTLA4, PD-1, or a PD-1 ligand. In some embodiments, the prior therapy is an inhibitor of CTLA4, such as ipilimumab. In some embodiments, the prior therapy is an inhibitor of PD-1, such as an anti-PD-1 antibody. In some embodiments, the prior therapy is an inhibitor of a PD-1 ligand (e.g., PD-L1), for example an anti-PD-L1 antibody.

In some embodiments according to any one of the methods described above, the cancer is liver cancer, a cancer of the digestive system (e.g., colon cancer, colorectal cancer), lung cancer, bone cancer, heart cancer, brain cancer, kidney cancer, bladder cancer, a hematological cancer (e.g., leukemia), skin cancer, breast cancer, thyroid cancer, pancreatic cancer, a head and/or neck cancer, an eye-related cancer, a male reproductive system cancer (e.g., prostate cancer, testicular cancer), or a female reproductive system cancer (e.g., uterine cancer, cervical cancer). In some embodiments, the cancer is a solid cancer. In some embodiments, the cancer is urothelial carcinoma. In some embodiments, the cancer is renal cell carcinoma. In some embodiments, the cancer is pancreatic cancer. In some embodiments, the cancer is advanced-stage cancer. In some embodiments, the cancer is metastatic cancer. In some embodiments, the cancer is Kaposi's sarcoma. In one embodiment, cancer includes but is not limited to colorectal cancer, gastric cancer, gastroesophageal junction cancer, esophageal cancer, endometrial cancer, or head and neck cancer. In another embodiment, cancer includes but is not limited to melanoma, non-small cell lung cancer (NSCLC), small cell lung cancer (SCLC), head and neck squamous cell cancer (HNSCC), classical Hodgkin lymphoma (cHL), primary mediastinal large B-cell lymphoma (PMBCL), urothelial carcinoma, microsatellite instability-high or mismatch repir deficient cancer, microsatellite instability-high or mismatch repair deficient colorectal cancer, gastric cancer, esophageal cancer, cervical cancer, hepatocellular carcinoma (HCC), Merkel cell carcinoma (MCC), renal cell carcinoma (RCC), endometrial carcinoma, tumor mutational burden-high (TMB-H) cancer, cutaneous squamous cell carcinoma (cSCC), or triple negative breast cancer (TNBC). The present disclosure is related to a method of treating cancer in a subject in need thereof, comprising administering to the subject a combination therapy comprising at least two pharmaceutical compositions.

In some embodiments, the anti-CTLA4 antibody and pembrolizumab are both administered intravenously. In some embodiments, the anti-CTLA4 antibody is administered subcutaneously. In some embodiments, the anti-CTLA4 antibody and pembrolizumab are administered intravenously or subcutaneously once every three weeks. In some embodiments, the anti-CTLA4 antibody and pembrolizumab administered intravenously or subcutaneously once every six weeks. In some embodiments, the subject receives at least 4 cycles of treatment with the anti-CTLA4 antibody and pembrolizumab. In some embodiments, the subject further receives a maintenance treatment comprising administering to the subject an effective amount of the anti-CTLA4 antibody about once every four weeks to about once every twelve weeks (e.g., once every 4, 6, 8, 10, or 12 weeks). In some embodiments, the doses of the anti-CTLA4 antibody and pembrolizumab may be administered at the same time. In other embodiments, the doses of the anti-CTLA4 antibody and pembrolizumab may be administered. For instance, pembrolizumab may be administered from about 0.5 hours to about 5 hours prior to or following administration of the anti-CTLA4 antibody on day 1 of the dosing schedule (e.g., three week dosing schedule).

In some embodiments according to any one of the methods described above, the subject is human.

It is to be understood that one, some, or all of the properties of the various embodiments described above and herein may be combined to form other embodiments of the present application. These and other aspects of the present application will become apparent to one of skill in the art. These and other embodiments of the present application are further described by the detailed description that follows.

### BRIEF DESCRIPTION OF FIGURES

FIG. 1 shows serum carcinoembryonic antigen (CEA) reduction in a MSS CRC patient with lung metastasis following combination treatment TY21580 and pembrolizumab.

FIG. 2 shows serum carcinoembryonic CEA reduction in a MSS CRC patient with liver and lung metastasis following combination treatment with TY21580 and pembrolizumab.

FIG. 3 shows modulation of immune biomarkers by combination treatment of TY21580 and pembrolizumab.

FIG. 4 shows TY21580 serum PK when dosed at 3 mg/kg Q3W in combination with pembrolizumab.

### DETAILED DESCRIPTION

### I. Definitions

Unless otherwise defined herein, scientific, and technical terms used in connection with the present application shall have the meanings that are commonly understood by those of ordinary skill in the art. Further, unless otherwise required by context, singular terms shall include pluralities and plural terms shall include the singular. Generally, nomenclatures used in connection with, and techniques of, antibody engineering, immunotherapy, cell and tissue culture, molecular biology, immunology, microbiology, genetics and protein and nucleic acid chemistry described herein are those well-known and commonly used in the art.

The term “antibody” is used herein in the broadest sense and specifically covers monoclonal antibodies (including full length monoclonal antibodies), polyclonal antibodies, multispecific antibodies (e.g., bispecific antibodies, trispecific antibodies), and antibody fragments (e.g., Fab, Fab′, Fab′-SH, F(ab′)₂, Fv and/or a single-chain variable fragment or scFv) so long as they exhibit the desired biological activity.

An “antibody fragment” or “antigen-binding fragment” refers to a molecule other than an intact antibody that comprises a portion of an intact antibody that binds the antigen to which the intact antibody binds. The antibody fragment retains the ability to bind specifically to the antigen bound by the full-length antibody, e.g. fragments that retain one or more CDR regions, e.g. all six CDRs. Examples of antibody fragments include but are not limited to Fv, Fab, Fab′, Fab′-SH, F(ab′)2; diabodies; linear antibodies; single-chain antibody molecules (e.g., scFv); and multispecific antibodies formed from antibody fragments

In some embodiments, the term “antibody” refers to an antigen-binding protein (i.e., immunoglobulin) having a basic four-polypeptide chain structure consisting of two identical heavy (H) chains and two identical light (L) chains. Each L chain is linked to an H chain by one covalent disulfide bond, while the two H chains are linked to each other by one or more disulfide bonds depending on the H chain isotype. Each heavy chain has, at the N-terminus, a variable region (abbreviated herein as V_{H}) followed by a constant region. The heavy chain constant region is comprised of three domains, C_{H1}, C_{H2} and C_{H3}. Each light chain has, at the N-terminus, a variable region (abbreviated herein as V_{I}) followed by a constant region at its other end. The light chain constant region is comprised of one domain, C_{L}. The V_{L} is aligned with the V_{H} and the C_{L} is aligned with the first constant domain of the heavy chain (CH1). The pairing of a V_{H} and V_{L} together forms a single antigen-binding site. An IgM antibody consists of 5 of the basic heterotetramer units along with an additional polypeptide called J chain, and therefore contains 10 antigen binding sites, while secreted IgA antibodies can polymerize to form polyvalent assemblages comprising 2-5 of the basic 4-chain units along with J chain.

The V_{H} and V_{L} regions can be further subdivided into regions of hypervariability, termed hyper-variable regions (HVR) based on structural and sequence analysis. HVRs are interspersed with regions that are more conserved, termed framework regions (FW) (see e.g., Chen et al. (1999) J. Mol. Biol. (1999) 293, 865-881). Each V_{H} and V_{L} is composed of three HVRs and four FWs, arranged from amino-terminus to carboxy-terminus in the following order: FW-1_HVR-1_FW-2_HVR-2_FW-3_HVR-3_FW4. Throughout the present application, the three HVRs of the heavy chain are referred to as HVR-H1, HVR-H2, and HVR-H3. Similarly, the three HVRs of the light chain are referred to as HVR-L1, HVR-L2, and HVR-L3.

As used herein, the term “CDR” or “CDRs” means complementarity determining region(s) in an immunoglobulin variable region or the non-contiguous antigen combining sites found within the variable region of both heavy and light chain polypeptides. As used herein, CDRs are defined using the Kabat numbering system, unless otherwise indicated. See e.g., Kabat et al., J. Biol. Chem. 252:6609-6616 (1977); Kabat et al., U.S. Dept. of Health and Human Services, “Sequences of proteins of immunological interest” (1991); Chothia et al., J. Mol. Biol. 196:901-917 (1987); Al-Lazikani B. et al., J. Mol. Biol., 273:927-948 (1997); MacCallum et al., J. Mol. Biol. 262:732-745 (1996); Abhinandan and Martin, Mol. Immunol., 45:3832-3839 (2008); Lefranc M. P. et al., Dev. Comp. Immunol., 27:55-77 (2003); and Honegger and Plückthun, J. Mol. Biol., 309:657-670 (2001), where the definitions include overlapping or subsets of amino acid residues when compared against each other. Nevertheless, application of either definition to refer to a CDR of an antibody or grafted antibodies or variants thereof is intended to be within the scope of the term as defined and used herein. CDR prediction algorithms and interfaces are known in the art, including, for example, Abhinandan and Martin, Mol. Immunol., 45:3832-3839 (2008); Ehrenmann F. et al., Nucleic Acids Res., 38: D301-D307 (2010); and Adolf-Bryfogle J. et al., Nucleic Acids Res., 43: D432-D438 (2015). The contents of the references cited in this paragraph are incorporated herein by reference in their entireties for use in the present invention and for possible inclusion in one or more claims herein.

The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. Within light and heavy chains, the variable and constant regions are joined by a “J” region of about 12 or more amino acids, with the heavy chain also including a “D” region of about 10 or more amino acids (see e.g., Fundamental Immunology Ch. 7 (Paul, W., ed., 2^{nd} ed. Raven Press, N.Y). (1989)).

The L chain from any vertebrate species can be assigned to one of two clearly distinct types, called kappa and lambda, based on the amino acid sequences of their constant domains. Depending on the amino acid sequence of the constant domain of their heavy chains (CH), antibodies can be assigned to different classes or isotypes. There are five classes of antibodies: IgA, IgD, IgE, IgG, and IgM, having heavy chains designated α (alpha), δ (delta), ε (epsilon), γ (gamma), and μ (mu), respectively. The IgG class of antibody can be further classified into four subclasses IgG1, IgG2, IgG3, and IgG4 by the gamma heavy chains, Y1-Y4, respectively.

The term “CTLA4” is used in the present application, and includes the human CTLA4 (e.g., UniProt accession number P16410), as well as variants, isoforms, and species homologs thereof (e.g., mouse CTLA4 (UniProt accession number P09793), rat CTLA4 (UniProt accession number Q9Z1A7), dog CTLA4 (UniProt accession number Q9XSI1), cynomolgus monkey CTLA4 (UniProt accession number G7PL88), etc.). Accordingly, an anti-CTLA4 antibody, as defined and disclosed herein, may also bind CTLA4 from species other than human. In other cases, an anti-CTLA4 antibody may be completely specific for the human CTLA4 and may not exhibit species or other types of cross-reactivity.

The term “CTLA4 antibody” refers to an antibody, as defined herein, capable of binding to human CTLA4.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256:495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352:624-628 and Marks et al. (1991) J. Mol. Biol. 222:581-597, for example. See also Presta (2005) J. Allergy Clin. Immunol. 116:731.

“PD-1 antagonist” means any chemical compound or biological molecule that blocks binding of PD-L1 expressed on a cancer cell to PD-1 expressed on an immune cell (T cell, B cell or Natural Killer T cell) and in specific embodiments also blocks binding of PD-L2 expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. In any of the treatment method, medicaments and uses of the present invention in which a human individual is being treated, the PD-1 antagonist blocks binding of human PD-L1 to human PD-1, and in specific embodiments blocks binding of both human PD-L1 and PD-L2 to human PD-1. Human PD-1 amino acid sequences can be found in NCBI Locus No.: NP_005009. Human PD-L1 and PD-L2 amino acid sequences can be found in NCBI Locus No.: NP_054862 and NP_079515, respectively.

“Pembrolizumab” (formerly known as MK-3475, SCH 900475 and lambrolizumab) alternatively referred to herein as “pembro,” is a humanized IgG4 mAb with the structure described in WHO Drug Information, Vol. 27, No. 2, pages 161-162 (2013) and which comprises the heavy and light chain amino acid sequences and CDRs described in Table B. Pembrolizumab has been approved by the U.S. FDA as described in the Prescribing Information for KEYTRUDA® (Merck Sharp & Dohme LLC, Rahway, NJ, USA, initial U.S. approval 2014, updated March 2021).

As used herein, a “pembrolizumab variant” or “a variant thereof” pertaining to a pembrolizumab sequence means a monoclonal antibody that comprises heavy chain and light chain sequences that are substantially identical to those in pembrolizumab, except for having three, two or one conservative amino acid substitutions at positions that are located outside of the light chain CDRs and six, five, four, three, two or one conservative amino acid substitutions that are located outside of the heavy chain CDRs, e.g., the variant positions are located in the FR regions or the constant region, and optionally has a deletion of the C-terminal lysine residue of the heavy chain. In other words, pembrolizumab and a pembrolizumab variant comprise identical CDR sequences, but differ from each other due to having a conservative amino acid substitution at no more than three or six other positions in their full length light and heavy chain sequences, respectively. A pembrolizumab variant is substantially the same as pembrolizumab with respect to the following properties: binding affinity to PD-1 and ability to block the binding of each of PD-L1 and PD-L2 to PD-1.

The term “epitope” refers to a part of an antigen to which an antibody (or antigen-binding fragment thereof) binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope can include various numbers of amino acids in a unique spatial conformation. Methods of determining spatial conformation of epitopes include, for example, x-ray crystallography, 2-dimensional nuclear magnetic resonance, deuterium and hydrogen exchange in combination with mass spectrometry, or site-directed mutagenesis, or all methods used in combination with computational modeling of antigen and its complex structure with its binding antibody and its variants (see e.g., Epitope Mapping Protocols in Methods in Molecular Biology, Vol. 66, G. E. Morris, Ed. (1996)). Once a desired epitope of an antigen is determined, antibodies to that epitope can be generated, e.g., using the techniques described herein. The generation and characterization of antibodies may also elucidate information about desirable epitopes. From this information, it is then possible to competitively screen antibodies for binding to the same epitope. An approach to achieve this is to conduct cross-competition studies to find antibodies that competitively bind with one another, i.e., the antibodies compete for binding to the antigen. A high throughput process for “binning” antibodies based upon their cross-competition is described in PCT Publication No. WO 03/48731.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see e.g., Flatman et al., J. Chromatogr. B 848:79-87 (2007).

As used herein, “sequence identity” between two polypeptide sequences indicates the percentage of amino acids that are identical between the sequences. The amino acid sequence identity of polypeptides can be determined conventionally using known computer programs such as Bestfit, FASTA, or BLAST (see e.g., Pearson, Methods Enzymol. 183:63-98 (1990); Pearson, Methods Mol. Biol. 132:185-219 (2000); Altschul et al., J. Mol. Biol. 215:403-410 (1990); Altschul et al., Nucelic Acids Res. 25:3389-3402 (1997)). When using Bestfit or any other sequence alignment program to determine whether a particular sequence is, for instance, 95% identical to a reference amino acid sequence, the parameters are set such that the percentage of identity is calculated over the full length of the reference amino acid sequence and that gaps in homology of up to 5% of the total number of amino acid residues in the reference sequence are allowed. This aforementioned method in determining the percentage of identity between polypeptides is applicable to all proteins, fragments, or variants thereof disclosed herein.

As used herein, the term “binds”, “binds to”, “specifically binds” “specifically binds to” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of ≤1 μM, ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding. An antibody that “specifically binds to” a specified target protein is an antibody that exhibits preferential binding to that target as compared to other proteins, but this specificity does not require absolute binding specificity. An antibody is considered “specific” for its intended target if its binding is determinative of the presence of the target protein in a sample, e.g. without producing undesired results such as false positives. Antibodies, or binding fragments thereof, useful in the present invention will bind to the target protein with an affinity that is at least two fold greater, preferably at least ten times greater, more preferably at least 20-times greater, and most preferably at least 100-times greater than the affinity with non-target proteins. As used herein, an antibody is said to bind specifically to a polypeptide comprising a given amino acid sequence, e.g. the amino acid sequence of a mature human PD-1 or human PD-L1 molecule, if it binds to polypeptides comprising that sequence but does not bind to proteins lacking that sequence

The term “treat”, “treating”, or “treatment”, with reference to a certain disease condition in a mammal, refers causing a desirable or beneficial effect in the mammal having the disease condition. The desirable or beneficial effect may include reduced frequency or severity of one or more symptoms of the disease (i.e., tumor growth and/or metastasis, or other effect mediated by the numbers and/or activity of immune cells, and the like), or arrest or inhibition of further development of the disease, condition, or disorder. In the context of treating cancer in a mammal, the desirable or beneficial effect may include inhibition of further growth or spread of cancer cells, death of cancer cells, inhibition of reoccurrence of cancer, reduction of pain associated with the cancer, or improved survival of the mammal. The effect can be either subjective or objective. For example, if the mammal is human, the human may note improved vigor or vitality or decreased pain as subjective symptoms of improvement or response to therapy. Alternatively, the clinician may notice a decrease in tumor size or tumor burden based on physical exam, laboratory parameters, tumor markers or radiographic findings. Some laboratory signs that the clinician may observe for response to treatment include normalization of tests, such as white blood cell count, red blood cell count, platelet count, erythrocyte sedimentation rate, and various enzyme levels. Additionally, the clinician may observe a decrease in a detectable tumor marker. Alternatively, other tests can be used to evaluate objective improvement, such as sonograms, nuclear magnetic resonance testing and positron emissions testing.

The term “prevent” or “preventing,” with reference to a certain disease condition in a mammal, refers to preventing or delaying the onset of the disease, or preventing the manifestation of clinical or subclinical symptoms thereof.

As used herein, a “subject”, “patient”, or “individual” may refer to a human or a non-human animal. A “non-human animal” may refer to any animal not classified as a human, such as domestic, farm, or zoo animals, sports, pet animals (such as dogs, horses, cats, cows, etc.), as well as animals used in research. Research animals may refer without limitation to nematodes, arthropods, vertebrates, mammals, frogs, rodents (e.g., mice or rats), fish (e.g., zebrafish or pufferfish), birds (e.g., chickens), dogs, cats, and non-human primates (e.g., rhesus monkeys, cynomolgus monkeys, chimpanzees, etc.). In some embodiments, the subject, patient, or individual is a human.

An “effective amount” refers to at least an amount effective, at dosages and for periods of time necessary, to achieve one or more desired or indicated effects, including a therapeutic or prophylactic result. An effective amount can be provided in one or more administrations. For purposes of the present application, an effective amount of antibody, drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition (e.g., an effective amount as administered as a monotherapy or combination therapy). Thus, an “effective amount” may be considered in the context of administering one or more therapeutic agents, and a single agent may be considered to be given in an effective amount if, in conjunction with one or more other agents, a desirable result may be or is achieved.

The terms “recurrence,” “relapse” or “relapsed” refers to the return of a cancer or disease after clinical assessment of the disappearance of disease. A diagnosis of distant metastasis or local recurrence can be considered a relapse.

The term “refractory” or “resistant” refers to a cancer or disease that has not responded to treatment.

As used herein, “complete response” or “CR” refers to disappearance of all target lesions; “partial response” or “PR” refers to at least a 30% decrease in the sum of the longest diameters (SLD) of target lesions, taking as reference the baseline SLD; and “stable disease” or “SD” refers to neither sufficient shrinkage of target lesions to qualify for PR, nor sufficient increase to qualify for PD, taking as reference the smallest SLD since the treatment started.

As used herein, “progressive disease” or “PD” refers to at least a 20% increase in the SLD of target lesions, taking as reference the smallest SLD recorded since the treatment started or the presence of one or more new lesions.

As used herein, “progression free survival” (PFS) refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “overall response rate” (ORR) refers to the sum of complete response (CR) rate and partial response (PR) rate.

As used herein, “overall survival” refers to the percentage of individuals in a group who are likely to be alive after a particular duration of time.

As used herein, a “baseline level” or “baseline value” refers to a level or a value of a subject before the subject begins a treatment, such as an anti-CTLA4 antibody treatment.

A “reference sample”, “reference cell”, “reference tissue”, “control sample”, “control cell”, or “control tissue”, as used herein, refers to a sample, cell, tissue, standard, or level that is used for comparison purposes. In one embodiment, a reference sample, reference cell, reference tissue, control sample, control cell, or control tissue is obtained from a healthy and/or non-diseased part of the body (e.g., tissue or cells) of the same subject or individual. For example, healthy and/or non-diseased cells or tissue adjacent to the diseased cells or tissue (e.g., cells or tissue adjacent to a tumor). In another embodiment, a reference sample is obtained from an untreated tissue and/or cell of the body of the same subject or individual. In yet another embodiment, a reference sample, reference cell, reference tissue, control sample, control cell, or control tissue is obtained from a healthy and/or non-diseased part of the body (e.g., tissues or cells) of an individual who is not the subject or individual. In even another embodiment, a reference sample, reference cell, reference tissue, control sample, control cell, or control tissue is obtained from an untreated tissue and/or cell of the body of an individual who is not the subject or individual.

An “effective response” of a patient or a patient's “responsiveness” to treatment with a medicament and similar wording refers to the clinical or therapeutic benefit imparted to a patient at risk for, or suffering from, a disease or disorder, such as cancer. In one embodiment, such benefit includes any one or more of: extending survival (including overall survival and progression free survival); resulting in an objective response (including a complete response or a partial response); or improving signs or symptoms of cancer.

A patient who “does not have an effective response” to treatment refers to a patient who does not have any one of extending survival (including overall survival and progression free survival); resulting in an objective response (including a complete response or a partial response); or improving signs or symptoms of cancer.

The methods and techniques of the present application are generally performed according to methods well known in the art and as described in various general and more specific references that are cited and discussed throughout the present specification unless otherwise indicated. Such references include, e.g., Sambrook and Russell, Molecular Cloning, A Laboratory Approach, Cold Spring Harbor Press, Cold Spring Harbor, N.Y. (2001), Ausubel et al., Current Protocols in Molecular Biology, John Wiley & Sons, NY (2002), and Harlow and Lane Antibodies: A Laboratory Manual, Cold Spring Harbor Laboratory Press, Cold Spring Harbor, N.Y. (1990). Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

As used herein, the twenty conventional amino acids and their abbreviations follow conventional usage. See Immunology—A Synthesis (2nd Edition, E. S. Golub and D. R. Gren, Eds., Sinauer Associates, Sunderland, Mass. (1991)).

As used herein, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the present application described herein include “comprising,” “consisting,” and “consisting essentially of” aspects and embodiments.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

### II. Methods of Treatment

The present application provides methods for treating cancers in a subject using an anti-CTLA4 antibody that specifically binds to human CTLA4. Any one of the anti-CTLA4 antibodies (including full-length antibodies and antigen-binding fragments thereof) in Section III “Anti-CTLA4 Antibodies” may be used in the methods described herein.

In some embodiments, there is provided a method of treating a cancer in a subject, wherein the cancer is resistant or refractory to an inhibitor of CTLA-4, PD-1 or a PD-1 ligand (e.g., PD-L1 or PD-L2), comprising administering to the subject an effective amount of an anti-CTLA4 antibody in combination with pembrolizumab, wherein the antibody comprises: (a) a heavy chain variable region comprising an HVR-H1 comprising the amino acid sequence of SEQ ID NO: 23, an HVR-H2 comprising the amino acid sequence of SEQ ID NO: 35, and an HVR-H3 comprising the amino acid sequence of SEQ ID NO: 45, and/or a light chain variable region comprising an HVR-L1 comprising the amino acid sequence of SEQ ID NO: 58, an HVR-L2 comprising the amino acid sequence of SEQ ID NO: 66, and an HVR-L3 comprising the amino acid sequence of SEQ ID NO: 75. In some embodiments, the cancer is resistant or refractory to an anti-PD-1 antibody. In some embodiments, the cancer is resistant or refractory to a different anti-CTLA4 antibody, such as ipilimumab. In some embodiments, the cancer is resistant or refractory to an anti-PD-L1 antibody. In some embodiments, the cancer is a solid cancer, such as advanced-stage and/or metastatic cancer. In some embodiments, the antibody comprises a heavy chain variable region comprising the amino acid sequence of SEQ ID NO: 87 or an amino acid sequence having at least 90% sequence identity to the amino acid sequence of SEQ ID NO: 87, and/or a light chain variable region comprising the amino acid sequence of SEQ ID NO: 100 or an amino acid sequence having at least 90% sequence identity to the amino acid sequence of SEQ ID NO: 100. In some embodiments, the antibody comprises a human IgG1 Fc region, such as a wildtype IgG1 Fc region or a variant that has enhanced ADCC activity. In some embodiments, the antibody is TY21580.

In some embodiments, there is provided a method of treating a cancer in a subject, comprising administering to the subject an effective amount of an anti-CTLA4 antibody as disclosed herein in combination with pembrolizumab, wherein the anti-CTLA4 antibody is administered at a dose of from about 3 mg/kg to about 10 mg/kg. In some embodiments, the anti-CTLA4 is administered at a dose of about 3 mg/kg. In some embodiments, the anti-CTLA4 antibody is administered at a dose of about 5 mg/kg. In some embodiments, the anti-CTLA4 antibody is administered at a dose of about 6 mg/kg. In some embodiments, the anti-CTLA4 antibody is administered at a dose of about 8 mg/kg. In some embodiments, the anti-CTLA4 antibody is administered at a dose of about 10 mg/kg.

In some embodiments, the cancer is resistant or refractory to an inhibitor of CTLA-4, PD-1 or a PD-1 ligand (e.g., PD-L1 or PD-L2). In some embodiments, the cancer is a solid cancer, such as advanced-stage and/or metastatic Cancer treatments can be evaluated by, e.g., tumor regression, tumor weight or size shrinkage, time to progression, duration of survival, progression free survival, overall response rate, duration of response, quality of life, protein expression and/or activity. Approaches to determining efficacy of therapy can be employed, including for example, measurement of response through radiological imaging.

The anti-CTLA4 antibodies and compositions provided by the present disclosure can be administered via any suitable enteral route or parenteral route of administration. The term “enteral route” of administration refers to the administration via any part of the gastrointestinal tract. Examples of enteral routes include oral, mucosal, buccal, and rectal route, or intragastric route. “Parenteral route” of administration refers to a route of administration other than enteral route. Examples of parenteral routes of administration include intravenous, intramuscular, intradermal, intraperitoneal, intratumor, intravesical, intraarterial, intrathecal, intracapsular, intraorbital, intracardiac, transtracheal, intraarticular, subcapsular, subarachnoid, intraspinal, epidural and intrasternal, subcutaneous, or topical administration. The antibodies and compositions of the disclosure can be administered using any suitable method, such as by oral ingestion, nasogastric tube, gastrostomy tube, injection, infusion, implantable infusion pump, and osmotic pump. The suitable route and method of administration may vary depending on a number of factors such as the specific antibody being used, the rate of absorption desired, specific formulation or dosage form used, type or severity of the disorder being treated, the specific site of action, and conditions of the patient, and can be readily selected by a person skilled in the art. In some embodiments, the anti-CTLA4 antibody is administered intravenously.

The effective amount of the anti-CTLA4 antibody may be administered in a single dose or in multiple doses. For methods that comprise administration of the anti-CTLA4 antibody in multiple doses, exemplary dosing frequencies include, but are not limited to weekly, weekly without break, weekly for two out of three weeks, weekly for three out of four weeks, once every three weeks, once every two weeks, monthly, every six months, yearly, etc. In some embodiments, the anti-CTLA4 antibody is administered about weekly, once every 2 weeks, once every 3 weeks, once every 6 weeks, or once every 12 weeks. In some embodiments, the intervals between each administration are less than about any of 3 years, 2 years, 12 months, 11 months, 10 months, 9 months, 8 months, 7 months, 6 months, 5 months, 4 months, 3 months, 2 months, 1 month, 4 weeks, 3 weeks, 2 weeks, or 1 week. In some embodiments, the intervals between each administration are more than about any of 1 week, 2 weeks, 3 weeks, 4 weeks, 1 month, 2 months, 3 months, 4 months, 5 months, 6 months, 7 months, 8 months, 9 months, 10 months, 11 months, 12 months, 2 years, or 3 years. In some embodiments, there is no break in the dosing schedule.

In some embodiments, the anti-CTLA4 antibody is administered at a low frequency, for example, any one of no more frequent than once per week, once every other week, once per three weeks, once per month, once per 2 months, once per 3 months, once per 4 months, once per 5 months, once per 6 months, once per 7 months, once per 8 months, once per 9 months, once per 10 months, once per 11 months, once per year, or less. In some embodiments, the anti-CTLA4 antibody is administered in a single dose. In some embodiments, the anti-CTLA4 antibody is administered about once every three weeks.

In some embodiments, the anti-CTLA4 antibody is administered for 2 or more cycles, such as about any one of 2, 3, 4, 5, 6, 7, 8, 9, 10, 11, 12, or more cycles. In some embodiments, the anti-CTLA4 antibody is administered for at least 4 cycles.

The anti-CTLA4 antibodies can be administered to patients in combination with pembrolizumab at doses that achieve high levels of receptor (CTLA-4) occupancy, and hence are efficacious while at the same time having minimal side effects. Hence, the anti-CTLA4 antibodies of the disclosure show improved therapeutic indexes relative to anti-CTLA4 antibodies such as Ipilimumab. For instance, in one embodiment, wherein the antibody comprises a heavy chain variable region comprising the amino acid sequence of SEQ ID NO: 87 and light chain variable region comprising the amino acid sequence of SEQ ID NO: 100, the anti-CTLA4 antibody can be administered as a single dose (in combination with pembrolizumab) that achieves greater than 50% receptor occupancy three weeks or even six weeks following administration. In some such embodiments, the anti-CTLA4 antibody can be administered as a single dose (in combination with pembrolizumab) that achieves greater than 60% receptor occupancy three weeks following administration. In other such embodiments, the anti-CTLA4 antibody can be administered as a single dose (in combination with pembrolizumab) that achieves greater than 70% receptor occupancy three weeks following administration. In other such embodiments, the anti-CTLA4 antibody can be administered as a single dose (in combination with pembrolizumab) that achieves greater than 80% receptor occupancy three weeks following administration. In other such embodiments, the anti-CTLA4 antibody can be administered as a single dose (in combination with pembrolizumab) that achieves from about 50% to about 80% receptor occupancy three weeks following administration. In other such embodiments, the anti-CTLA4 antibody can be administered as a single dose (in combination with pembrolizumab) that achieves from about 60% to about 75% receptor occupancy three weeks following administration. In other such embodiments, the anti-CTLA4 antibody can be administered as a single dose (in combination with pembrolizumab) that achieves greater than 60% receptor occupancy six weeks following administration. In other such embodiments, the anti-CTLA4 antibody can be administered as a single dose (in combination with pembrolizumab) that achieves greater than 70% receptor occupancy six weeks following administration. In other such embodiments, the anti-CTLA4 antibody can be administered as a single dose (in combination with pembrolizumab) that achieves from about 50% to about 70% receptor occupancy six weeks following administration.

In some embodiments, the treatment comprises an initial phase and a subsequent maintenance phase. In some embodiments, the anti-CTLA4 antibody is administered less frequently in the maintenance phase than in the initial phase. In some embodiments, the anti-CTLA4 antibody is administered at the same frequently in the maintenance phase as in the initial phase. In some embodiments, the treatment comprises an initial phase wherein the anti-CTLA4 antibody is administered about once every three weeks for at least 4 cycles, and a maintenance phase wherein the anti-CTLA4 antibody is administered about once every 4 weeks to once every 12 weeks, such as once every 4 weeks, once every 6 weeks, once every 8 weeks, once every 10 weeks, or once every 12 weeks. In some embodiments, the dosing frequency in the maintenance phase is adjusted depending on one or more biomarkers, such as T_{reg} cells, CD8+ Tₑₘ cells, CD4+ Tₑₘ cells, a ratio of CD8+ Tₑₘ cells to T_{reg} cells, a ratio of CD4+ Tₑₘ cells to T_{reg} cells, and/or NK cells. For example, if the subject shows an increase in the ratio of CD8+ Tₑₘ cells to T_{reg} cells after receiving the anti-CTLA4 antibody, the subject may be further administered an anti-CTLA4 antibody at about every 4 weeks.

The administration of the anti-CTLA4 antibody in combination with pembrolizumab can be extended over an extended period of time, such as from about a week to about a month, from about a month to about a year, from about a year to about several years. In some embodiments, the anti-CTLA4 antibody is administered over a period of at least any of about 1 week, 2 weeks, 3 weeks, 4 weeks, 5 weeks, 1 month, 2 months, 3 months, 4 months, 5 months, 6 months, 7 months, 8 months, 9 months, 10 months, 11 months, 12 months, 1 year, 2 years, 3 years, 4 years, or more.

The methods described herein are useful for treating a variety of cancers. In some embodiments, the cancer is a solid cancer. In some embodiments, the cancer is a liquid cancer. A variety of cancers where CTLA4 is implicated, whether malignant or benign and whether primary or secondary, may be treated or prevented with a method provided by the disclosure. Exemplary cancers include, but are not limited to, liver cancer, a cancer of the digestive system (e.g., colon cancer, colorectal cancer), lung cancer, bone cancer, heart cancer, brain cancer, kidney cancer, bladder cancer, a hematological cancer (e.g., leukemia), skin cancer, breast cancer, thyroid cancer, pancreatic cancer, a head and/or neck cancer, an eye-related cancer, a male reproductive system cancer (e.g., prostate cancer, testicular cancer), or a female reproductive system cancer (e.g., uterine cancer, cervical cancer). In some embodiments, the cancer is kidney cancer, such as renal cell carcinoma, or urothelial carcinoma. In some embodiments, the cancer is a cold tumor. In some embodiments, the cancer is resistant or refractory to one or more prior therapies, such as immunotherapies, including immune checkpoint inhibitor(s). In some embodiments, the cancer is a tumor that T cells cannot penetrate because the tumor has not been recognized by the immune system, or provoked an immune response.

In some embodiments, the anti-CTLA4 antibodies of the disclosure can be used to treat colorectal cancer (CRC). In some embodiments, the colorectal cancer is microsatellite stable (MSS)-colorectal cancer. In some embodiments, the colorectal cancer has metalized to other organs, such as the lung or the liver. In some embodiments, the colorectal cancer patient had previously been treated with other chemotherapeutic reagents. Such chemotherapeutic reagents include, but are not limited to, FOLFOX, FOLFIRI/Avastin, Erbitux, Lonsurf, IO-202, APN401, or IPH5201.

In some embodiments, the anti-CTLA4 antibodies of the disclosure can be used to treat Kaposi's sarcoma.

In some embodiments, the anti-CTLA4 antibodies of the disclosure can be used to treat head and neck squamous cell carcinoma (HNSCC).

In some embodiments, the anti-CTLA4 antibodies of the disclosure can be used to treat pancreatic cancer.

In some embodiments, the anti-CTLA4 antibodies of the disclosure can be used to treat ovarian cancer.

In some embodiments, the subject has been previously treated with a prior therapy. In some embodiments, the subject has previously received any one of 1, 2, 3, 4, or more prior therapies. In some embodiments, the subject has exhausted all other available therapies. In some embodiments, the subject is unresponsive or resistant to a prior therapy. In some embodiments, the subject has disease reoccurrence subsequent to a prior therapy. In some embodiments, the subject is refractory to a prior therapy. In some embodiments, the subject has failed a prior therapy within about 1 year, 6 months, 3 months or less. In some embodiments, the subject has not previously received a prior therapy.

In some embodiments, the subject has been previously treated with a standard therapy for the cancer. In some embodiments, the subject is unresponsive or resistant to a standard therapy. In some embodiments, the subject has disease reoccurrence subsequent to a standard therapy. In some embodiments, the subject is refractory to a standard therapy. In some embodiments, the subject has failed a standard therapy within about 1 year, 6 months, 3 months or less. In some embodiments, the subject has not previously received a standard therapy. In some embodiments, the subject has refused or is ineligible for a standard therapy.

In some embodiments, the prior therapy (e.g., standard therapy) is selected from the group consisting of viral gene therapy, immunotherapy, targeted therapy, radiation therapy, and chemotherapy. In some embodiments, the prior therapy is an immune checkpoint inhibitor. In some embodiments, the prior therapy is an inhibitor of CTLA4, PD-1, or a PD-1 ligand (e.g., PD-L1 or PD-L2). In some embodiments, the prior therapy is an inhibitor of CTLA4, such as an anti-CTLA4 antibody that is different from the anti-CTLA4 antibodies described herein. In some embodiments, the prior therapy is ipilimumab.

In some embodiments, the prior therapy is an inhibitor of PD-1 or a PD-1 ligand, including a PD-1 binding antagonist, a PDL1 binding antagonist and a PDL2 binding antagonist. Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the prior therapy is an inhibitor of PD-1 that is a molecule that inhibits the binding of PD-1 to its ligand binding partners. In some embodiments, the inhibitor of a PD-1 ligand is an inhibitor of PD-L1 and/or PD-L2. In some embodiments, the inhibitor of PD-L1 is a molecule that inhibits the binding of PDL1 to its binding partners. In some embodiments, a PD-L2 binding partner is PD-1 and/or B7-1. In some embodiments, the inhibitor of a PD-1 ligand is a molecule that inhibits the binding of PD-L2 to its binding partners. In some embodiments, a PD-L2 binding partner is PD-1. The inhibitor may be an antibody, an antigen binding fragment thereof, an immunoadhesin, a fusion protein, or oligopeptide.

In some embodiments, the prior therapy is an anti-PD-1 antibody selected from pembrolizumab, 2E5 (Cstone Pharmaceuticals), tislelizumab (BGB-A317), BGB-108, STI-A1110, AM0001, BI 754091, sintilimab (IBI308), cetrelimab (JNJ-63723283), toripalimab (JS-001), camrelizumab (SHR-1210, INCSHR-1210, HR-301210), MEDI-0680 (AMP-514), MGA-012 (INCMGA 0012), nivolumab (BMS-936558, MDX1106, ONO-4538), spartalizumab (PDR001), PF-06801591, cemiplimab (REGN-2810, REGEN2810), dostarlimab (TSR-042, ANB011), pidilizumab (CT-011), FITC-YT-16 (PD-1 binding peptide), APL-501 or CBT-501 or genolimzumab (GB-226), AB-122, AK105, AMG 404, BCD-100, F520, HLX10, HX008, JTX-4014, LZM009, Sym021, PSB205, AMP-224 (fusion protein targeting PD-1), CX-188 (PD-1 probody), AGEN-2034, GLS-010, budigalimab (ABBV-181), AK-103, BAT-1306, CS-1003, AM-0001, TILT-123, BH-2922, BH-2941, BH-2950, ENUM-244C8, ENUM-388D4, HAB-21, H EISCOI 11-003, IKT-202, MCLA-134, MT-17000, PEGMP-7, PRS-332, RXI-762, STI-1110, VXM-10, XmAb-23104, AK-112, HLX-20, SSI-361, AT-16201, SNA-01, AB122, PD1-PIK, PF-06936308, RG-7769, CAB PD-1 Abs, AK-123, MEDI-3387, MEDI-5771, 4H1128Z-E27, REMD-288, SG-001, BY-24.3, CB-201, IBI-319, ONCR-177, Max-1, CS-4100, JBI-426, CCC-0701, CCX-4503, biosimilars thereof, or derivatives thereof. In some embodiments, the anti-PD-1 antibody is selected from the group consisting of nivolumab, and CT-011. In some embodiments, the inhibitor of PD-1 is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the inhibitor of PD-1 is AMP-224. In some embodiments, the anti-PD-1 antibody is nivolumab (CAS Registry Number: 946414-94-4). Nivolumab, also known as MDX-1106-04, MDX-1106, ONO-4538, BMS-936558, and OPDIVO®, is an anti-PD-1 antibody described in WO2006/121168. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Prior therapies (e.g., standard therapies) also encompass surgery to remove a tumor and radiation therapy. Exemplary radiation therapies include, but are not limited to, ionizing (electromagnetic) radiotherapy (e.g., X-rays or gamma rays) and particle beam radiation therapy (e.g., high linear energy radiation). The source of radiation can be external or internal to the subject.

The methods described herein are useful for various aspects of cancer treatment. In some embodiments, there is provided a method of inhibiting cell proliferation (such as tumor growth) in an individual, comprising administering to the individual an effective amount of any one of the anti-CTLA4 antibodies described herein in combination with pembrolizumab. In some embodiments, at least about 10% (including for example at least about any of 20%, 30%, 40%, 60%, 70%, 80%, 90%, 95% or more) cell proliferation is inhibited.

In some embodiments, there is provided a method of inhibiting tumor metastasis in an individual, comprising administering to the individual an effective amount of any one of the anti-CTLA4 antibodies described herein in combination with pembrolizumab. In some embodiments, at least about 10% (including for example at least about any of 20%, 30%, 40%, 60%, 70%, 80%, 90%, 95% or more) metastasis is inhibited.

In some embodiments, there is provided a method of reducing (such as eradicating) pre-existing tumor metastasis (such as metastasis to the lymph node) in an individual, comprising administering to the individual an effective amount of any one of the anti-CTLA4 antibodies described herein in combination with pembrolizumab. In some embodiments, at least about 10% (including for example at least about any of 20%, 30%, 40%, 60%, 70%, 80%, 90%, 95% or more) metastasis is reduced.

In some embodiments, there is provided a method of reducing incidence or burden of preexisting tumor metastasis (such as metastasis to the lymph node) in an individual, comprising administering to the individual an effective amount of any one of the anti-CTLA4 antibodies described herein in combination with pembrolizumab.

In some embodiments, there is provided a method of reducing tumor size in an individual, comprising administering to the individual an effective amount of any one of the anti-CTLA4 antibodies described herein in combination with pembrolizumab. In some embodiments, the method reduces tumor size by at least about 10% (including for example at least about any of 20%, 30%, 40%, 60%, 70%, 80%, 90%, 95% or more).

In some embodiments, there is provided a method of prolonging time to disease progression of cancer in an individual, comprising administering to the individual an effective amount of any one of the anti-CTLA4 antibodies described herein in combination with pembrolizumab. In some embodiments, the method prolongs the time to disease progression by at least any of 1, 2, 3, 4, 5, 6, 7, 8, 9, 10, 11, 12, 16, 20, 24, 28, 32, 36, or more weeks.

In some embodiments, there is provided a method of prolonging survival (e.g., overall survival or progression-free survival) of an individual having cancer, comprising administering to the individual an effective amount of any one of the anti-CTLA4 antibodies described herein in combination with pembrolizumab. In some embodiments, the method prolongs the survival of the individual by at least any of 1, 2, 3, 4, 5, 6, 7, 8, 9, 10, 11, 12, 18, or 24 months.

In some embodiments, there is provided a method of alleviating one or more symptoms in an individual having cancer, comprising administering to the individual an effective amount of any one of the anti-CTLA4 antibodies described herein in combination with pembrolizumab.

In some embodiments, there is provided a method of improving the quality of life in an individual having cancer, comprising administering to the individual an effective amount of any one of the anti-CTLA4 antibodies described herein in combination with pembrolizumab.

The anti-CTLA4 antibody and pembrolizumab may be in combination with one or more additional therapeutic agents or therapies. In some embodiment, anti-CTLA4 antibody and pembrolizumab is administered in combination with one or more additional therapeutic agents for separate, sequential or simultaneous administration. The term “additional therapeutic agent” refers to any therapeutic agent other than an anti-CTLA4 antibody provided by the disclosure. In some embodiments, there is provided a combination therapy for treating cancer in a subject, which comprises administering to the subject a therapeutically effective amount of an anti-CTLA4 antibody described herein in combination with one or more additional therapeutic agents. In some embodiments, anti-CTLA4 antibody is administered in combination with one or more additional therapeutic agents comprising chemotherapeutic agents, immunotherapeutic agents, and/or hormone therapeutic agents. In some embodiments, the one or more additional therapeutic agents are selected from the group consisting of selected from the group consisting of viral gene therapy, immune checkpoint inhibitors, targeted therapies, radiation therapies, and chemotherapies.

### III. Anti-CTLA4 Antibodies

The method described herein comprise administration of an anti-CTLA4 antibody that specifically binds to human CTLA4, including CTLA4 antibodies, antigen-binding fragments of the CTLA4 antibodies, and derivatives of the CTLA4 antibodies. Exemplary anti-CTLA4 antibodies have been described, for example, in International Publication No. WO2019149281A1, which is incorporated herein by reference in its entirety.

In some embodiments, the anti-CTLA4 antibody is any one of the antibodies described herein, including antibodies described with reference to specific amino acid sequences of HVRs, variable regions (VL, VH), and light and heavy chains (e.g., IgG1, IgG2, IgG4). In some embodiments, the antibodies are human antibodies. In some embodiments, the antibodies are humanized antibodies and/or chimeric antibodies.

In some embodiments, the antibodies or antigen-binding fragments described herein have antagonist activity on human CTLA4. In some embodiments, the antibodies or antigen-binding fragments repress one or more activities of human CTLA4 when a cell (e.g., a human cell) expressing human CTLA4 is contacted by the antibody or antigen binding fragment (e.g., CTLA4 blockade as measured by an increase in a reporter gene signal using a CLA4 blockage reporter gene assay).

In some embodiments, the antibodies or antigen-binding fragments are cross-reactive with monkey (e.g., cynomolgus monkey), mouse, rat, and/or dog CTLA4. In some embodiments, the antibodies or antigen-binding fragments are cross-reactive with monkey CTLA4. In some embodiments, the antibodies or antigen-binding fragments are cross-reactive with mouse CTLA4. In some embodiments, the antibodies or antigen-binding fragments are cross-reactive with rat CTLA4. In some embodiments, the antibodies or antigen-binding fragments are cross-reactive with dog CTLA4. In some embodiments, the antibodies or antigen binding fragments are cross reactive with monkey and mouse CTLA4; monkey and rat CTLA4; monkey and dog CTLA4; mouse and rat CTLA4; mouse and dog CTLA4; rat and dog CTLA4; monkey, mouse, and rat CTLA4; monkey, mouse, and dog CTLA4; monkey, rat, and dog CTLA4; mouse, rat, and dog CTLA4; or monkey, mouse, rat, and dog CTLA4. In some embodiments, the antibodies or antigen binding fragments are cross-reactive if the antibodies or antigen-binding fragments binds to a non-human CTLA4 molecule with a K_{D} less than about 500 nM (e.g., less than about 1 nM, less than about 10 nM, less than about 25 nM, less than about 50 nM, less than about 75 nM, less than about 100 nM, less than about 150 nM, less than about 200 nM, less than about 250 nM, less than about 300 nM, less than about 350 nM, etc.). Methods of measuring antibody cross-reactivity are known in the art, including, without limitation, surface plasmon resonance, an ELISA, isothermal titration calorimetry, a filter binding assay, an EMSA, etc. In some embodiments, the cross-reactivity is measured by ELISA.

In some embodiments, the antibodies induce ADCC effects against a CTLA4 expressing cell (e.g., against CTLA4-expressing human cells such as Tregs) after the antibody binds to the cell-expressed CTLA4. Methods of measuring ADCC effects (e.g., in vitro methods) are known in the art. In some embodiments, the antibodies induce ADCC effects by more than about 10% (e.g., induce ADCC by more than about 10%, more than about 15%, more than about 20%, more than about 25%, more than about 30%, more than about 35%, more than about 40%, etc.) relative to a control (e.g., an isotype control or ipilimumab).

In some embodiments, the antibodies or antigen-binding fragments are capable of inhibiting tumor cell growth and/or proliferation. In some embodiments, the tumor cell growth and/or proliferation is inhibited by at least about 5% (e.g., at least about 5%, at least about 10%, at least about 20%, at least about 30%, at least about 40%, at least about 50%, at least about 60%, at least about 70%, at least about 80%, at least about 90%, or at least about 99%) when contacted with the antibodies or antigen-binding fragments relative to corresponding tumor cells not contacted with the antibodies or antigen-binding fragments (or relative to corresponding tumor cells contacted with an isotype control antibody). In some embodiments, the antibodies or antigen-binding fragments are capable of reducing tumor volume in a subject when the subject is administered the antibodies or antigen-binding fragments. In some embodiments, the antibodies or antigen-binding fragments are capable of reducing tumor volume in a subject by at least about 5% (e.g., at least about 5%, at least about 10%, at least about 20%, at least about 30%, at least about 40%, at least about 50%, at least about 60%, at least about 70%, at least about 80%, at least about 90%, or at least about 99%) relative to the initial tumor volume in the subject (e.g., prior to administration of the antibodies or antigen-binding fragments; as compared to a corresponding tumor in a subject administered an isotype control antibody). Methods of monitoring tumor cell growth/proliferation, tumor volume, and/or tumor inhibition are known in the art.

In some embodiments, the antibodies or antigen-binding fragments have therapeutic effect on a cancer. In some embodiments, the antibodies or antigen-binding fragments reduce one or more signs or symptoms of a cancer. In some embodiments, a subject suffering from a cancer goes into partial or complete remission when administered the antibodies or antigen-binding fragments.

In some embodiments, the antibodies or antigen-binding fragments block the binding between CTLA4 and one or more of its binding partners (e.g., human CTLA4 and human CD80, human CTLA4 and human CD86). In some embodiments, the antibodies or antigen-binding fragments block the binding between CTLA4 and its ligand in vitro. In some embodiments, the antibody or antigen-binding fragment has a half maximal inhibitory concentration (IC₅₀) of about 500 nM or less (e.g., about 500 nM or less, about 400 nM or less, about 300 nM or less, about 200 nM or less, about 100 nM or less, about 50 nM or less, about 25 nM or less, about 10 nM or less, about 1 nM or less, etc.) for blocking binding of CTLA4 to CD80 and/or CD86. In some embodiments, the antibody or antigen-binding fragment has a half maximal inhibitory concentration (IC₅₀) of about 100 nM or less for blocking binding of CTLA4 to CD80 and/or CD86. In some embodiments, the antibody or antigen-binding fragment completely blocks binding of human CTLA4 to CD80 and/or CD86 when provided at a concentration of about 100 nM or greater (e.g., about 100 nM or greater, about 500 nM or greater, about 1 μM or greater, about 10 μM or greater, etc.). As used herein, the term “complete blocking” or “completely blocks” refers to the antibody or antigen-binding fragment's ability to reduce binding between a first protein and a second protein by at least about 80% (e.g., at least about 80%, at least about 85%, at least about 90%, at least about 95%, at least about 99%, etc.). Methods of measuring the ability of an antibody or antigen-binding fragment to block binding of a first protein (e.g., human CTLA4) and a second protein (e.g., human CD80 or human CD86) are known in the art, including, without limitation, via BIAcore analysis, ELISA assays, and flow cytometry. In some embodiments, the anti-CTLA4 antibodies described herein have lower activity in blocking ligand binding than ipilimumab.

In some embodiments, the anti-CTLA4 antibody binds human CTLA4 with a K_{D} of 1000 nM or less (e.g., 50 nM or less, 10 nM or less) as measured by surface plasmon resonance. In some embodiments, the antibody is cross-reactive with at least one non-human species selected from cynomolgus monkey, mouse, rat, and dog.

In some embodiments, the anti-CTLA4 antibody comprising a heavy chain variable region and a light chain variable region, wherein the heavy chain variable region comprises an HVR-H1, an HVR-H2, and an HVR-H3, and the light chain variable region comprises an HVR-L1, an HVR-L2, and an HVR-L3, wherein the HVR-H1 comprises an amino acid sequence according to a formula YSISSGYHWSWI (SEQ ID NO: 23), the HVR-H2 comprises an amino acid sequence according to a formula LARIDWDDDKYYSTSLKSRL (SEQ ID NO: 35), the HVR-H3 comprises an amino acid sequence according to a formula ARSYVYFDY (SEQ ID NO: 45), the HVR-L1 comprises an amino acid sequence according to a formula RASQSVRGRFLA (SEQ ID NO: 58), the HVR-L2 comprises an amino acid sequence according to a formula DASNRATGI (SEQ ID NO: 66), and the HVR-L3 comprises an amino acid sequence according to a formula YCQQSSSWPPT (SEQ ID NO: 75).

In some embodiments, the antibody comprises: a) a heavy chain variable region comprising an amino acid sequence of SEQ ID NO: 87 and b) a light chain variable region comprising an amino acid sequence of SEQ ID NOS: 100 (Table A). In some embodiments, the antibody comprises a heavy chain variable region comprising an amino acid sequence having at least 90% (e.g., at least 90%, 91%, 92%, 93%, 94%, 95%, 96%, 97%, 98%, or 99%) sequence identity to a sequence of SEQ ID NOS: 87, and/or a light chain variable region comprising an amino acid sequence having at least 90% (e.g., at least 90%, 91%, 92%, 93%, 94%, 95%, 96%, 97%, 98%, or 99%) sequence identity to a sequence selected from SEQ ID NO: 100.

TABLE A
Anti-CTLA4 variable region amino acid sequences

| Ab | | |
|---|---|---|
| name: | VH: | VL: |
| TY21580 | EVQLVESGGGLVQPGGSLRLSCAASGYSISSG | DIQLTQSPSSLSASVGDRVTITCRASQSVRG |
| | YHWSWIRQAPGKGLEWLARIDWDDDKYYST | RFLAWYQQKPGKAPKLLIYDASNRATGIPS |
| | SLKSRLTISRDNSKNTLYLQLNSLRAEDTAVYY | RFSGSGSGTDFTLTISSLQPEDFATYYCQQS |
| | CARSYVYFDYWGQGTLVTVSS | SSWPPTFGQGTKVEIKR |
| | (SEQ ID NO: 87) | (SEQ ID NO: 100) |

The CTLA4 antibodies described herein may be in any class, such as IgG, IgM, IgE, IgA, or IgD. In some embodiments, the CTLA4 antibodies are in the IgG class, such as IgG1, IgG2, IgG3, or IgG4 subclass. A CTLA4 antibody can be converted from one class or subclass to another class or subclass using methods known in the art. An exemplary method for producing an antibody in a desired class or subclass comprises the steps of isolating a nucleic acid encoding a heavy chain of a CTLA4 antibody and a nucleic acid encoding a light chain of a CTLA4 antibody, isolating the sequence encoding the V_{H} region, ligating the V_{H} sequence to a sequence encoding a heavy chain constant region of the desired class or subclass, expressing the light chain gene and the heavy chain construct in a cell, and collecting the CTLA4 antibody. Antibodies of the present application may be monoclonal antibodies or polyclonal antibodies. Antibodies of the present application may be monospecific antibodies or multispecific (e.g., bispecific, trispecific, etc.) antibodies. In some embodiments, the CTLA4 antibodies described herein may include one or more Fc mutations (e.g., that modulate (increase or decrease) ADCC or CDC activities). Any suitable Fc mutations known in the art may be used in the CTLA4 antibodies of the present application.

In some embodiments, the anti-CTLA4 antibody comprises a heavy chain comprising the amino acid sequence: EVOLVESGGGLVQPGGSLRLSCAASGYSISSGYHWSWIRQAPGKGLEWLARIDWDDDKYYSTSL KSRLTISRDNSKNTLYLOLNSLRAEDTAVYYCARSYVYFDYWGQGTLVTVSSASTKGPSVFPLA PSSKSTSGGTAALGCLVKDYFPEPVTVSWNSGALTSGVHTFPAVLQSSGLYSLSSVVTVPSSSL GTQTYICNVNHKPSNTKVDKKVEPKSCDKTHTCPPCPAPELLGGPSVFLFPPKPKDTLMISRTP EVTCVVVDVSHEDPEVKFNWYVDGVEVHNAKTKPREEQYNSTYRVVSVLTVLHQDWLNGKEYKC KVSNKALPAPIEKTISKAKGQPREPQVYTLPPSRDELTKNQVSLTCLVKGFYPSDIAVEWESNG QPENNYKTTPPVLDSDGSFFLYSKLTVDKSRWQQGNVFSCSVMHEALHNHYTQKSLSLSPG (SEQ ID NO:125) and a light chain comprising the amino acid sequence DIQLTQSPSSLSASVGDRVTITCRASQSVRGRFLAWYQQKPGKAPKLLIYDASNRATGIPSRFS GSGSGTDFTLTISSLOPEDFATYYCOQSSSWPPTFGQGTKVEIKRTVAAPSVFIFPPSDEQLKS GTASVVCLLNNFYPREAKVOWKVDNALQSGNSQESVTEQDSKDSTYSLSSTLTLSKADYEKHKV YACEVTHQGLSSPVTKSENRGEC (SEQ ID NO:127). In some embodiments, the anti-CTLA4 antibody comprises a heavy chain comprising the amino acid sequence EVOLVESGGGLVQPGGSLRLSCAASGYSISSGYHWSWIRQAPGKGLEWLARIDWDDDKYYSTSL KSRLTISRDNSKNTLYLQLNSLRAEDTAVYYCARSYVYFDYWGQGTLVTVSSASTKGPSVFPLA PSSKSTSGGTAALGCLVKDYFPEPVTVSWNSGALTSGVHTFPAVLQSSGLYSLSSVVTVPSSSL GTQTYICNVNHKPSNTKVDKKVEPKSCDKTHTCPPCPAPELLGGPSVFLFPPKPKDTLMISRTP EVTCVVVDVSHEDPEVKFNWYVDGVEVHNAKTKPREEQYNSTYRVVSVLTVLHQDWLNGKEYKC KVSNKALPAPIEKTISKAKGQPREPQVYTLPPSRDELTKNQVSLTCLVKGFYPSDIAVEWESNG QPENNYKTTPPVLDSDGSFFLYSKLTVDKSRWQQGNVFSCSVMHEALHNHYTQKSLSLSPGK (SEQ ID NO:126) and a light chain comprising the amino acid sequence DIQLTQSPSSLSASVGDRVTITCRASQSVRGRFLAWYQQKPGKAPKLLIYDASNRATGIPSRFS GSGSGTDFTLTISSLOPEDFATYYCQQSSSWPPTFGQGTKVEIKRTVAAPSVFIFPPSDEQLKS GTASVVCLLNNFYPREAKVOWKVDNALQSGNSQESVTEQDSKDSTYSLSSTLTLSKADYEKHKV YACEVTHQGLSSPVTKSENRGEC (SEQ ID NO:127). In some embodiments, the anti-CTLA4 antibody refers to a mix of antibody species, wherein each antibody species has a light chain comprising the amino acid sequence of SEQ ID NO: 127 and a heavy chain comprising either the amino acid sequence of SEQ ID NO: 125 or 126.

In some embodiments, the anti-CTLA4 antibody is an antigen-binding fragment of an anti-CTLA4 antibody, the antigen-binding fragments of a CTLA4 antibody include: (i) a Fab fragment, which is a monovalent fragment consisting of the V_{L}, V_{H}, C_{L} and C_{H}1 domains; (ii) a F(ab′)₂ fragment, which is a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody; (v) a dAb fragment (Ward et al., (1989) Nature 341:544-546), which consists of a V_{H} domain; (vi) an isolated CDR, and (vii) single chain antibody (scFv), which is a polypeptide comprising a V_{L} region of an antibody linked to a V_{H} region of an antibody (see e.g., Bird et al. (1988) Science 242:423-426; Huston et al. (1988) Proc. Natl. Acad. Sci. USA 85:5879-5883).

In some embodiments, the anti-CTLA4 antibody is a derivative of any one of the anti-CTLA4 antibodies described herein. In some embodiments, the antibody derivative is derived from modifications of the amino acid sequences of an illustrative antibody (e.g., a “parent antibody”) of the present application while conserving the overall molecular structure of the parent antibody amino acid sequence. Amino acid sequences of any regions of the parent antibody chains may be modified, such as framework regions, HVR regions, or constant regions. Types of modifications include substitutions, insertions, deletions, or combinations thereof, of one or more amino acids of the parent antibody.

In some particular embodiments, the derivative comprises 1, 2, 3, 4, 5, 6, 7, 8, 9, 10, 11, 12, 13, 14, or 15 conservative or non-conservative substitutions, and/or 1, 2, 3, 4, 5, 6, 7, 8, 9, 10, 11, 12, 13, 14, or 15 additions and/or deletions to an amino acid sequence as set forth above.

Amino acid substitutions encompass both conservative substitutions and non-conservative substitutions. The term “conservative amino acid substitution” means a replacement of one amino acid with another amino acid where the two amino acids have similarity in certain physico-chemical properties such as polarity, charge, solubility, hydrophobicity, hydrophilicity, and/or the amphipathic nature of the residues involved. For example, substitutions typically may be made within each of the following groups: (a) nonpolar (hydrophobic) amino acids, such as alanine, leucine, isoleucine, valine, proline, phenylalanine, tryptophan, and methionine; (b) polar neutral amino acids, such as glycine, serine, threonine, cysteine, tyrosine, asparagine, and glutamine; (c) positively charged (basic) amino acids, such as arginine, lysine, and histidine; and (d) negatively charged (acidic) amino acids, such as aspartic acid and glutamic acid. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 1 below:

TABLE 1
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

“Framework region” or “FR” as used herein means the immunoglobulin variable regions excluding the CDR regions.

The modifications may be made in any positions of the amino acid sequences of the antibody, including the HVRs, framework regions, or constant regions. In one embodiment, the present application provides an antibody derivative that contains the V_{H} and V_{L} HVR sequences of an illustrative antibody of this disclosure, yet contains framework sequences different from those of the illustrative antibody. Such framework sequences can be obtained from public DNA databases or published references that include germline antibody gene sequences. For example, germline DNA sequences for human heavy and light chain variable region genes can be found in the Genbank database or in the “VBase” human germline sequence database (Kaba et al., Sequences of Proteins of Immunological Interest, Fifth Edition, U.S. Department of Health and Human Services, NIH Publication No. 91-3242 (1991); Tomlinson et al., J. Mol. Biol. 227:776-798 (1992); and Cox et al., Eur. J. Immunol. 24:827-836 (1994)). Framework sequences that may be used in constructing an antibody derivative include those that are structurally similar to the framework sequences used by illustrative antibodies of the disclosure For example, the HVR-H1, HVR-H2, and HVR-H3 sequences, and the HVR-L1, HVR-L2, and HVR-L3 sequences of an illustrative antibody can be grafted onto framework regions that have the identical sequence as that found in the germline immunoglobulin gene from which the framework sequence derive, or the HVR sequences can be grafted onto framework regions that contain one or more mutations as compared to the germline sequences.

In some embodiments, the antibody derivative is a chimeric antibody, which comprises an amino acid sequence of an illustrative antibody of the disclosure. In one example, one or more HVRs from one or more illustrative antibodies are combined with HVRs from an antibody from a non-human animal, such as mouse or rat. In another example, all of the HVRs of the chimeric antibody are derived from one or more illustrative antibodies. In some particular embodiments, the chimeric antibody comprises one, two, or three HVRs from the heavy chain variable region and/or one, two, or three HVRs from the light chain variable region of an illustrative antibody. Chimeric antibodies can be generated using conventional methods known in the art.

Another type of modification is to mutate amino acid residues within the HVR regions of the V_{H} and/or V_{L} chain. Site-directed mutagenesis or PCR-mediated mutagenesis can be performed to introduce the mutation(s) and the effect on antibody binding, or other functional property of interest, can be evaluated in in vitro or in vivo assays known in the art. Typically, conservative substitutions are introduced. The mutations may be amino acid additions and/or deletions. Moreover, typically no more than one, two, three, four or five residues within an HVR region are altered. In some embodiments, the antibody derivative comprises 1, 2, 3, or 4 amino acid substitutions in the heavy chain HVRs and/or in the light chain HVRs. In another embodiment, the amino acid substitution is to change one or more cysteines in an antibody to another residue, such as, without limitation, alanine or serine. The cysteine may be a canonical or non-canonical cysteine. In one embodiment, the antibody derivative has 1, 2, 3, or 4 conservative amino acid substitutions in the heavy chain HVR regions relative to the amino acid sequences of an illustrative antibody.

Modifications may also be made to the framework residues within the V_{H} and/or V_{L} regions. Typically, such framework variants are made to decrease the immunogenicity of the antibody. One approach is to “back mutate” one or more framework residues to the corresponding germline sequence. An antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “back mutated” to the germline sequence by, for example, site-directed mutagenesis or PCR-mediated mutagenesis.

In addition, modifications may also be made within the Fc region of an illustrative antibody, typically to alter one or more functional properties of the antibody, such as serum half-life, complement fixation, Fc receptor binding, and/or antigen-dependent cellular cytotoxicity. In one example, the hinge region of CH1 is modified such that the number of cysteine residues in the hinge region is altered, e.g., increased or decreased. This approach is described further in U.S. Pat. No. 5,677,425. The number of cysteine residues in the hinge region of CH1 is altered to, for example, facilitate assembly of the light and heavy chains or to increase or decrease the stability of the antibody. In another case, the Fc hinge region of an antibody is mutated to decrease the biological half-life of the antibody.

Furthermore, an antibody of the present application may be modified to alter its potential glycosylation site or pattern in accordance with routine experimentation known in the art. In another aspect, the present application provides a derivative of a CTLA4 antibody that contains at least one mutation in a variable region of a light chain or heavy chain that changes the pattern of glycosylation in the variable region. Such an antibody derivative may have an increased affinity and/or a modified specificity for binding an antigen. The mutations may add a novel glycosylation site in the V region, change the location of one or more V region glycosylation site(s), or remove a pre-existing V region glycosylation site. In one embodiment, the present application provides a derivative of a CTLA4 antibody having a potential N-linked glycosylation site at asparagine in the heavy chain variable region, wherein the potential N-linked glycosylation site in one heavy chain variable region is removed. In another embodiment, the present application provides a derivative of a CTLA4 antibody having a potential N-linked glycosylation site at asparagine in the heavy chain variable region, wherein the potential N-linked glycosylation site in both heavy chain variable regions is removed. Method of altering the glycosylation pattern of an antibody is known in the art, such as those described in U.S. Pat. No. 6,933,368, the disclosure of which incorporated herein by reference.

### IV. PD-1 Antagonist

In one embodiment, the PD-1 antagonist useful in the treatment, medicaments and uses of the present invention include a monoclonal antibody (mAb), or antigen binding fragment thereof, that specifically binds to PD-1 or PD-L1, and preferably specifically binds to human PD-1 or human PD-L1. The mAb may be a human antibody, a humanized antibody or a chimeric antibody, and may include a human constant region. In some embodiments the human constant region is selected from the group consisting of IgG1, IgG2, IgG3 and IgG4 constant regions, and in some embodiments, the human constant region is an IgG1 or IgG4 constant region. In some embodiments, the antigen binding fragment is selected from the group consisting of Fab, Fab′-SH, F(ab′)2, scFv and Fv fragments.

Examples of mAbs that bind to human PD-1, and useful in the treatment method, medicaments and uses of the present invention, are described in U.S. patent nos. U.S. Pat. Nos. 7,488,802, 7,521,051, 8,008,449, 8,354,509, and 8,168,757, and International application publn. nos. WO2004/004771, WO2004/072286, WO2004/056875, US2011/0271358, and WO 2008/156712. Specific anti-human PD-1 mAbs useful as the PD-1 antagonist in the treatment method, medicaments and uses of the present invention include: pembrolizumab (also known as MK-3475), a humanized IgG4 mAb with the structure described in WHO Drug Information, Vol. 27, No. 2, pages 161-162 (2013) and that comprises the heavy and light chain amino acid sequences shown in Table B; nivolumab (BMS-936558), a human IgG4 mAb with the structure described in WHO Drug Information, Vol. 27, No. 1, pages 68-69 (2013); the humanized antibodies h409A11, h409A16 and h409A17, which are described in WO2008/156712, and AMP-514, which is being developed by MedImmune; cemiplimab; camrelizumab; sintilimab; tislelizumab; and toripalimab. Additional anti-PD-1 antibodies contemplated for use herein include MEDI0680 (U.S. Pat. No. 8,609,089), BGB-A317 (U.S. Patent publ. no. 2015/0079109), INCSHR1210 (SHR-1210) (PCT International application publ. no. WO2015/085847), REGN-2810 (PCT International application publ. no. WO2015/112800), PDR001 (PCT International application publ. no. WO2015/112900), TSR-042 (ANB011) (PCT International application publ. no. WO2014/179664) and STI-1110 (PCT International application publ. no. WO2014/194302).

Examples of mAbs that bind to human PD-L1, and useful in the treatment method, medicaments and uses of the present invention, are described in U.S. Pat. No. 8,383,796. Specific anti-human PD-L1 mAbs useful as the PD-1 antagonist in the treatment method, medicaments and uses of the present invention include BMS-936559, MEDI4736, and MSB0010718C.

In some embodiments, the PD-1 antagonist is pembrolizumab ((KEYTRUDA®, Merck Sharp & Dohme LLC, Rahway, NJ, USA), nivolumab (OPDIVO™, Bristol-Myers Squibb Company, Princeton, NJ, USA), atezolizumab (TECENTRIQ™, Genentech, San Francisco, CA, USA), durvalumab (IMFINZI™, AstraZeneca Pharmaceuticals LP, Wilmington, DE), cemiplimab (LIBTAYO™, Regeneron Pharmaceuticals, Tarrytown, NY, USA) avelumab (BAVENCIO™, Merck KGaA, Darmstadt, Germany) or dostarlimab (JEMPERLI™, GlaxoSmithKline LLC, Philadelphia, PA). In other embodiments, the PD-1 antagonist is pidilizumab (U.S. Pat. No. 7,332,582), AMP-514 (MedImmune LLC, Gaithersburg, MD, USA), PDR001 (U.S. Pat. No. 9,683,048), BGB-A317 (U.S. Pat. No. 8,735,553), or MGA012 (MacroGenics, Rockville, MD).

In one embodiment, the PD-1 antagonist useful in the methods of the invention is an anti-PD-1 antibody that blocks the binding of PD-1 to PD-L1 and PD-L2. In some embodiments of the treatment methods, medicaments and uses of the present invention, the PD-1 antagonist is a monoclonal antibody, or antigen binding fragment thereof, that comprises: (a) a light chain variable region comprising light chain CDR1, CDR2 and CDR3 of SEQ ID NOs: 10, 11 and 12, respectively and (b) a heavy chain variable region comprising heavy chain CDR1, CDR2 and CDR3 of SEQ ID NOs: 15, 16 and 17, respectively.

In other embodiments of the treatment methods, medicaments and uses of the present invention, the PD-1 antagonist is a monoclonal antibody, or antigen binding fragment thereof, that specifically binds to human PD-1 and comprises (a) a heavy chain variable region comprising SEQ ID NO:18 or a variant thereof, and (b) a light chain variable region comprising SEQ ID NO:13 or a variant thereof. A variant of a heavy chain variable region sequence is identical to the reference sequence except having up to six conservative amino acid substitutions in the framework region (i.e., outside of the CDRs). A variant of a light chain variable region sequence is identical to the reference sequence except having up to three conservative amino acid substitutions in the framework region (i.e., outside of the CDRs).

In another embodiment of the treatment methods, medicaments and uses of the present invention, the PD-1 antagonist is a monoclonal antibody that specifically binds to human PD-1 and comprises (a) a heavy chain comprising SEQ ID NO: 19 and (b) a light chain comprising SEQ ID NO:14. In one embodiment, the PD-1 antagonist is an anti-PD-1 antibody that comprises two heavy chains and two light chains, and wherein the heavy and light chains comprise the amino acid sequences in SEQ ID NO:19 and SEQ ID NO:14, respectively.

In all of the above treatment methods, medicaments and uses, the PD-1 antagonist inhibits the binding of PD-L1 to PD-1, and in specific embodiments also inhibits the binding of PD-L2 to PD-1. In some embodiments of the above treatment methods, medicaments and uses, the PD-1 antagonist is a monoclonal antibody, or an antigen binding fragment thereof, that specifically binds to PD-1 or to PD-L1 and blocks the binding of PD-L1 to PD-1.

Table B below provides a list of the amino acid sequences of exemplary anti-PD-1 mAbs for use in the treatment method, medicaments and uses of the present invention.

TABLE B
Exemplary PD-1 Antibody Sequences

| | | |
|---|---|---|
| Antibody | | SEQ |
| Feature | Amino Acid Sequence | ID NO. |

Pembrolizumab Light Chain

| | | |
|---|---|---|
| CDR1 | RASKGVSTSGYSYLH | 10 |
| CDR2 | LASYLES | 11 |
| CDR3 | QHSRDLPLT | 12 |
| Variable | EIVLTQSPATLSLSPGERATLSCRASKGVSTSGYSYLHWY | 13 |
| Region | QQKPGQAPRLLIYLASYLESGVPARFSGSGSGTDFTLTISSL | |
| | EPEDFAVYYCQHSRDLPLTFGGGTKVEIK | |
| Light Chain | EIVLTQSPATLSLSPGERATLSCRASKGVSTSGYSYLHWY | 14 |
| | QQKPGQAPRLLIYLASYLESGVPARFSGSGSGTDFTLTISSL | |
| | EPEDFAVYYCQHSRDLPLTFGGGTKVEIKRTVAAPSVFIFP | |
| | PSDEQLKSGTASVVCLLNNFYPREAKVQWKVDNALQSGN | |
| | SQESVTEQDSKDSTYSLSSTLTLSKADYEKHKVYACEVTH | |
| | QGLSSPVTKSFNRGEC | |

Pembrolizumab Heavy Chain

| | | |
|---|---|---|
| CDR1 | NYYMY | 15 |
| CDR2 | GINPSNGGTNFNEKFKN | 16 |
| CDR3 | RDYRFDMGFDY | 17 |
| Variable | QVQLVQSGVEVKKPGASVKVSCKASGYTFTNYYMYWVR | 18 |
| Region | QAPGQGLEWMGGINPSNGGTNFNEKFKNRVTLTTDSSTT | |
| | TAYMELKSLQFDDTAVYYCARRDYRFDMGFDYWGQGTT | |
| | VTVSS | |
| Heavy Chain | QVQLVQSGVEVKKPGASVKVSCKASGYTFTNYYMYWVR | 19 |
| | QAPGQGLEWMGGINPSNGGTNFNEKFKNRVTLTTDSSTT | |
| | TAYMELKSLQFDDTAVYYCARRDYRFDMGFDYWGQGTT | |
| | VTVSSASTKGPSVFPLAPCSRSTSESTAALGCLVKDYFPEP | |
| | VTVSWNSGALTSGVHTFPAVLQSSGLYSLSSVVTVPSSSL | |
| | GTKTYTCNVDHKPSNTKVDKRVESKYGPPCPPCPAPEFLG | |
| | GPSVFLFPPKPKDTLMISRTPEVTCVVVDVSQEDPEVQFN | |
| | WYVDGVEVHNAKTKPREEQFNSTYRVVSVLTVLHQDWL | |
| | NGKEYKCKVSNKGLPSSIEKTISKAKGQPREPQVYTLPPSQ | |
| | EEMTKNQVSLTCLVKGFYPSDIAVEWESNGQPENNYKTT | |
| | PPVLDSDGSFFLYSRLTVDKSRWQEGNVFSCSVMHEALH | |
| | NHYTQKSLSLSLGK | |

TABLE C
Additional PD-1 Antibodies and Antigen Binding Fragments Useful
in the Formulations, Methods and Uses of the Invention.
A. Antibodies and antigen binding fragments comprising light
and heavy chain CDRs of hPD-1.08A in WO2008/156712

| | |
|---|---|
| CDRL1 | SEQ ID NO: 20 |
| CDRL2 | SEQ ID NO: 21 |
| CDRL3 | SEQ ID NO: 22 |
| CDRH1 | SEQ ID NO: 23 |
| CDRH2 | SEQ ID NO: 24 |
| CDRH3 | SEQ ID NO: 25 |

C. Antibodies and antigen binding fragments
comprising the mature h109A heavy chain variable
region and one of the mature K09A light chain variable
regions in WO 2008/156712

| | |
|---|---|
| Heavy chain VR | SEQ ID NO: 26 |
| Light chain VR | SEQ ID NO: 27 or SEQ ID NO: |
| | 28 or SEQ ID NO: 29 |

D. Antibodies and antigen binding fragments
comprising the mature 409 heavy chain and one of
the mature K09A light chains in WO 2008/156712

| | |
|---|---|
| Heavy chain | SEQ ID NO: 30 |
| Light chain | SEQ ID NO: 31 or SEQ ID NO: |
| | 32 or SEQ ID NO: 33 |

In one embodiment, the anti-PD-1 antibody or antigen-binding fragment thereof comprises a heavy chain constant region, e.g. a human constant region, such as g1, g2, g3, or g4 human heavy chain constant region or a variant thereof. In another embodiment, the anti-PD-1 antibody or antigen-binding fragment thereof comprises a light chain constant region, e.g. a human light chain constant region, such as lambda or kappa human light chain region or a variant thereof. By way of example, and not limitation, the human heavy chain constant region can be g4 and the human light chain constant region can be kappa. In an alternative embodiment, the Fc region of the antibody is g4 with a Ser228Pro mutation (Schuurman, J et. al., Mol. Immunol. 38:1-8, 2001). In some embodiments, different constant domains may be appended to humanized VL and VH regions derived from the CDRs provided herein. For example, if a particular intended use of an antibody (or fragment) of the present invention were to call for altered effector functions, a heavy chain constant domain other than human IgG1 may be used, or hybrid IgG1/IgG4 may be utilized. Although human IgG1 antibodies provide for long half-life and for effector functions, such as complement activation and antibody-dependent cellular cytotoxicity, such activities may not be desirable for all uses of the antibody. In such instances a human IgG4 constant domain, for example, may be used. The present invention includes the use of anti-PD-1 antibodies or antigen-binding fragments thereof which comprise an IgG4 constant domain. In one embodiment, the IgG4 constant domain can differ from the native human IgG4 constant domain (Swiss-Prot Accession No. P01861.1) at a position corresponding to position 228 in the EU system and position 241 in the KABAT system, where the native Ser108 is replaced with Pro, in order to prevent a potential inter-chain disulfide bond between Cys106 and Cys109 (corresponding to positions Cys 226 and Cys 229 in the EU system and positions Cys 239 and Cys 242 in the KABAT system) that could interfere with proper intra-chain disulfide bond formation. See Angal et al. (1993) Mol. Imunol. 30:105. In other instances, a modified IgG1 constant domain which has been modified to increase half-life or reduce effector function can be used.

In another embodiment, the PD-1 antagonist is an antibody or antigen binding protein that has a variable light domain and/or a variable heavy domain with at least 95%, 90%, 85%, 80%, 75% or 50% sequence identity to one of the variable light domains or variable heavy domains described above, and exhibits specific binding to PD-1. In another embodiment of the methods of treatment of the invention, the PD-1 antagonist is an antibody or antigen binding protein comprising variable light and variable heavy domains having up to 1, 2, 3, 4, or 5 or more amino acid substitutions, and exhibits specific binding to PD-1.

In some embodiments, pembrolizumab is administered at a dose of about 400 mg every 6 weeks.

In some embodiment, pembrolizumab is administered at a dose of about 2 mg/kg. In some embodiment, pembrolizumab is administered at a dose of about 2 mg/kg every three weeks. In particular embodiments, the patient is a pediatric patient.

In some embodiment, pembrolizumab is administered as a 30 minute (−5 minutes/+10 minutes) intravenous infusion. In one embodiment, the selected dose of pembrolizumab is administered by IV infusion over a time period of between 25 and 40 minutes, or about 30 minutes.

In one aspect, pembrolizumab in included in a pharmaceutical composition with a pharmaceutically acceptable carrier or diluent and may include additional pharmaceutically acceptable excipients.

### V. Pharmaceutical Compositions, Kits, and Articles of Manufacture

The anti-CTLA4 antibodies described herein and the pembrolizumab can be administered in a pharmaceutical compositions comprising a pharmaceutically acceptable carrier. The anti-CTLA4 antibody and the pembrolizumab can be administered in separate pharmaceutical compositions or in a single pharmaceutical composition. The compositions can be prepared by conventional methods known in the art.

The term “pharmaceutically acceptable carrier” refers to any inactive substance that is suitable for use in a formulation for the delivery of an active agent (e.g., the anti-CTLA4 antibody or pembrolizumab). A carrier may be an anti-adherent, binder, coating, disintegrant, filler or diluent, preservative (such as antioxidant, antibacterial, or antifungal agent), sweetener, absorption delaying agent, wetting agent, emulsifying agent, buffer, and the like. Examples of suitable pharmaceutically acceptable carriers include water, ethanol, polyols (such as glycerol, propylene glycol, polyethylene glycol, and the like) dextrose, vegetable oils (such as olive oil), saline, buffer, buffered saline, and isotonic agents such as sugars, polyalcohols, sorbitol, and sodium chloride. The compositions may be in any suitable forms, such as liquid, semi-solid, and solid dosage forms. Examples of liquid dosage forms include solution (e.g., injectable and infusible solutions), microemulsion, liposome, dispersion, or suspension. Examples of solid dosage forms include tablet, pill, capsule, microcapsule, and powder. A particular form of the composition suitable for delivering an anti-CTLA4 antibody is a sterile liquid, such as a solution, suspension, or dispersion, for injection or infusion. Sterile solutions can be prepared by incorporating the antibody in the required amount in an appropriate carrier, followed by sterilization microfiltration. Generally, dispersions are prepared by incorporating the antibody into a sterile vehicle that contains a basic dispersion medium and other carriers. In the case of sterile powders for the preparation of sterile liquid, methods of preparation include vacuum drying and freeze-drying (lyophilization) to yield a powder of the active ingredient plus any additional desired ingredient from a previously sterile-filtered solution thereof. The various dosage forms of the compositions can be prepared by conventional techniques known in the art.

In some embodiments, there is provided an article of manufacture comprising materials useful for the treatment of a cancer. The article of manufacture can comprise a container and a label or package insert on or associated with the container. Suitable containers include, for example, bottles, vials, syringes, etc. The containers may be formed from a variety of materials such as glass or plastic. Generally, the container holds a composition, which is effective for treating a cancer, described herein, and may have a sterile access port (for example, the container may be an intravenous solution bag or a vial having a stopper pierceable by a hypodermic injection needle). Package insert refers to instructions customarily included in commercial packages of therapeutic products that contain information about the indications, usage, dosage, administration, contraindications and/or warnings concerning the use of such therapeutic products. In some embodiments, the package insert indicates that the composition is used for treating a cancer. The label or package insert may further comprise instructions for administering the composition to a patient.

Additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for treatment of a cancer described herein, optionally in combination with the articles of manufacture. Kits of the present application include one or more containers comprising any one of the compositions described herein (or unit dosage form and/or article of manufacture). In some embodiments, the kit further comprises other agents (e.g., one or more additional therapeutic agents) and/or instructions for use in accordance with any of the methods described herein. The kit may further comprise a description of selection of individuals suitable for treatment. Instructions supplied in the kits of the present application are typically written instructions on a label or package insert (e.g., a paper sheet included in the kit), but machine-readable instructions (e.g., instructions carried on a magnetic or optical storage disk) are also acceptable.

For example, in some embodiments, there is provided a kit comprising a pharmaceutical composition comprising any one of the anti-CTLA4 antibodies described herein and a pharmaceutically acceptable carrier; pembrolizumab and a pharmaceutically acceptable carrier; and instructions for administering the pharmaceutical composition to a subject having a cancer. In some embodiments, the kit further comprises a pharmaceutical composition comprising an additional therapeutic agent, such as a chemotherapeutic agent. In some embodiments, the kit comprises one or more assays or reagents thereof for determining a level of one or more biomarkers described herein (e.g., CD8+ T cells, CD4+ T cells, CD8+ Tₑₘ cells, CD4+ Tₑₘ cells, T_{reg} cells, a ratio of CD8+ Tₑₘ cells to T_{reg} cells, a ratio of CD4+ Tₑₘ cells to T_{reg} cells, NK cells, B cells).

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The containers may be unit doses, bulk packages (e.g., multi-dose packages) or sub-unit doses. Kits may also include multiple unit doses of the pharmaceutical compositions and instructions for use and packaged in quantities sufficient for storage and use in pharmacies, for example, hospital pharmacies and compounding pharmacies.

### Examples

The invention can be further understood by reference to the following examples, which are provided by way of illustration and are not meant to be limiting.

Example 1. Phase Ib, Open-Label, Dose Escalation Study of TY21580 in Combination with Pembrolizumab (Anti-PD-1 Antibody) in Patients with Advanced/Metastatic Solid Tumors

The following example describes a phase Ib clinical trial to evaluate the safety, tolerability, PK, and preliminary efficacy of a TY21580-pembrolizumab combination regimen in patients with advanced/metastatic solid tumors. TY21580 is an anti-CTLA4 fully human IgG1 monoclonal antibody. TY21580 is administered IV over a period of 60-90 minutes. Pembrolizumab (KEYTRUDA®, Merck Sharp & Dohme LLC, Rahway, NJ, USA) is a PD-1 receptor-blocking antibody (a humanized IgG4 monoclonal antibody) which is indicated for the treatment of patients across a number of cancers. Pembrolizumab is administered IV over a period of 30 minutes. For the TY21580-pembrolizumab combination regimen, TY21580 is administered 30-60 minutes post end of infusion of pembrolizumab.

Objectives. Primary objectives of the study are to assess the safety and tolerability of TY21580 at escalating dose levels, TY21580 in combination with pembrolizumab in adults with advanced/metastatic solid tumors; to determine the maximum tolerated doses (MTDs); and to assess the preliminary antitumor activity of TY21580-pembrolizumab combination regimen. Secondary objectives of the study are to assess the pharmacokinetic (PK) profile of TY21580 and pembrolizumab; to assess the dose proportionality of key PK parameters (area under the time concentration curve [AUC], maximum [peak] serum concentration [Cmax], etc.); to assess the immunogenicity of TY21580 and pembrolizumab; to characterize the relationship between immunogenicity (anti-drug antibody [ADA] positivity) and PK, safety, and efficacy parameters; to assess the preliminary antitumor activity of TY21580-pembrolizumab regimen; to assess the safety and tolerability of TY21580 in combination with pembrolizumab in adults with advanced/metastatic solid tumors; to assess the PK profile of TY21580 and pembrolizumab; and to assess the immunogenicity of TY21580 and pembrolizumab. An exploratory objective of the study is to assess pharmacodynamic or potentially predictive biomarkers for TY21580, including but not limited to cytokines (IL-1β, IL-2, IL-6, interferon [IFN]-γ, and tumor necrosis factor [TNF]-α, etc.), serum proteins (sCTLA4, sPD-L1, sCD25, CXCL11, etc.), tumor infiltrating lymphocytes (TILs), regulatory T cells (Treg; CD4+ Tem, CD8+ Tem, Ki67, etc.), and other tissue biomarkers (MSI, TMB, PD-L1, etc.).

Study Design. This is a Phase 1b, open-label, multicenter, dose escalation and dose expansion study to evaluate the safety, tolerability, PK, and preliminary efficacy of a TY21580-pembrolizumab combination regimen in patients with advanced/metastatic solid tumors.

A modified Toxicity Probability Interval (mTPI) design with a target DLT rate of approximately 30% is applied for dose escalation and confirmation to determine the RP2D for TY21580 in combination with pembrolizumab. The dose escalation includes 4 dose escalation cohorts as in Table 2 below:

TABLE 2
TY21580-Pembrolizumab Dose Levels

| | Dose Level | TY21580 | Pembrolizumab |
|---|---|---|---|
| Design | (DL) | (mg/kg, Q3W) | (mg, Q3W) |
| mTPI design | DL1 | 3.0* | 200 |
| | DL2 | 6.0** | 200 |
| | DL3 | 10.0 | 200 |
| | DL4 | 20.0 | 200 |

DL = dose level;
mTPI = modified toxicity probability interval;
Q3W = every 3 weeks
*This might be adjusted based on dose-escalation of TY21580 in other clinical studies.
**This dose it to be determined based on clinical data from combination dose level 1 (DL1)

Starting from one level below the TY21580 monotherapy cleared dose, dose escalation of TY21580 in combination with a fixed dose of pembrolizumab continues to the next dose level, until the RP2D of the combination is determined. A de-escalation dose of TY21580 is available if the starting dose of TY21580 is deemed not tolerable. All dose escalation and de-escalation decisions are based on the occurrence of DLTs at a given dose during the first 21-day period (Cycle 1) and are made by a Safety Review Committee (SRC) comprised of the Principal Investigator (PI), the Medical Monitors, and the Sponsor.

For TY21580 combination treatment with pembrolizumab, both drugs are dosed Q3W for up to 35 cycles with pembrolizumab remaining constant at 200 mg for each dose level of TY21580 and in each cohort. The dose and dosing frequency of pembrolizumab will not be changed.

The safety and tolerability of each dose level are assessed by SRC after all patients enrolled in the dose level have been followed for at least 21 days after the first dose of TY21580-pembrolizumab combination regimen (DLT observation period).

Once either the MTD or maximum administered dose (MAD) are reached, the RP2D is defined. Any changes require submission of a revised protocol to the Independent Ethics Committee (IEC)/Institutional Review Board (IRB) and appropriate regulatory authorities. The RP2D will be decided based on the observation of either the MTD, or by the MAD in the absence of an MTD. Options for consideration for the RP2D will also include dose levels below the MTD or MAD, as well as intermediate doses between the pre-specified dose levels (e.g., between 3 and 20 mg/kg), based on an overall assessment of all safety data, as well as all available PK and pharmacodynamic data, and documented objective response observations during dose escalation.

A treatment cycle is 21 days with one IV dose of TY21580 in combination with pembrolizumab administered on Day 1. DLTs are evaluated during the initial 21 days. Toxicities are assessed using the National Cancer Institute Common Terminology Criteria for Adverse Events (NCI CTCAE) v5.0. Patients are treated on study with TY21580-pembrolizumab Q3W dosing regimen until documented confirmed disease progression according to RECIST v1.1 and/or iRECIST, development of significant toxicity, withdrawal of consent, or other discontinuation/withdrawal reason, or up to 35 cycles (Q3W), whichever occurs first. During the study, patients are evaluated for safety and toxicity, PK, immunogenicity, objective response, DOR, PFS, OS, and biomarkers.

Safety Evaluations. Safety assessments are carried out during specified periodic physical examination findings, vital signs, ECOG performance status, laboratory variables (e.g., liver tests/monitoring, hematology, coagulation tests, serum chemistry, urine tests and pregnancy test), ECG, and AEs. AEs are graded according to the NCI CTCAE v5.0. Investigators and site personnel are responsible for properly documenting and reporting AEs/SAEs. Safety Review Committee (SRC) consist of enrolling Investigators and Sponsor representatives. The SRC reviews available safety, clinical activity, PK, and pharmacodynamic data, and identifies any DLT at the completion of each dose level cohort and makes recommendations regarding dosing and dose escalation. The SRC may recommend an intermediate dose level to be evaluated, within the range of dose levels specified in the protocol. These decisions are documented.

Efficacy Evaluations. Tumor assessments for response/progression are performed at baseline, and every 6 weeks (±1 week) for the first 4 cycles. If treatment continues beyond 4 cycles, then assessments are carried out every 9 weeks (±1 week) for the remaining treatment duration thereafter, until disease progression or death, treatment/study discontinuation due to treatment toxicity, loss to follow-up, withdrawal of consent, start of new cancer treatment, or study completion/closure, whichever occurs first. Response and progression are evaluated in this study using the international criteria proposed by the revised RECIST v1.1 guideline(s), and/or iRECIST.

Pharmacokinetic and Immunogenicity Evaluations. Blood samples are collected from all patients during the first cycle to determine the serum concentration of TY21580 and pembrolizumab. PK parameters for TY21580 are monitored more intensively during the first treatment cycle. Reduced PK sampling is conducted for pembrolizumab. Noncompartmental analysis for TY21580 is conducted using WinNonlin 8.3 or higher versions. PK parameters include, but not limited to AUC_{0-21d}, AUCₗₐₛₜ, Cₘₐₓ, Tₘₐₓ, t_{1/2}, MRT, CL, V_{d} will be reported. Dose proportionality will also be assessed for AUC and Cₘₐₓ. Blood samples for ADAs against TY21580 are collected at pre-dose of cycles 1-4, and every 4 cycles thereafter if treatment continues beyond 4 cycles. Reduced ADA sampling is conducted for pembrolizumab. Additionally, ADA samples are collected at the end of the treatment and at 30 days when feasible after the last dose.

Pharmacodynamics Evaluations. Pharmacodynamic biomarkers for TY21580 are listed and summarized by protocol specified time point and treatment and include, but are not limited to: soluble proteins (sCTLA-4, MMPs), peripheral immune cell subset profiling, tumor infiltrated lymphocytes, and pharmacogenomics markers both in peripheral blood and tumor tissues (if available).

Optional Biopsy Evaluations. Biopsies from the patients before, during, and at the end of treatment is optional, but strongly recommended, for biomarker evaluation. Patients may have available sufficient and adequate formalin fixed tumor tissue sample (e.g., 15 FFPE slides) preferably from a biopsy of a tumor lesion obtained either at the time of or after the diagnosis of advanced disease has been made and from a site not previously irradiated. Alternatively, patients may have a biopsy taken prior to entering the study to provide adequate tissue. The index lesion should not be used for the optional biopsy. Patients with biopsy accessible tumors may also undergo optional post-treatment tumor biopsies at Cycle 3 and at end of treatment. Patients are given a separate, specific written consent to provide baseline, on-treatment, and/or end of treatment biopsies. Biopsies at Cycle 3 should be collected after radiographic tumor scans scheduled for that cycle have been completed.

### Interim Results

Six patients had been treated with TY21580 (3 mg/kg, Q3W)+Pembrolizumab (200 mg, Q3W) combination therapy. Patients were generally heavily pre-treated (Table 3). Tumor types consist of the breast, colon and pancreatic cancer, etc., and are generally considered as “cold” tumors.

TABLE 3
Baseline Characteristics of Patients

| Characteristics | N = 6 | |
|---|---|---|
| Age (years), median (range) | 61.5 | (47, 74) |
| Female, n (%) | 3 | (50%) |

Race, n (%)

| | | |
|---|---|---|
| Caucasian, n (%) | 5 | (83%) |
| Black or African American, n (%) | 1 | (17%) |

ECOG, n(%)

| | | |
|---|---|---|
| 0 | 2 | (33%) |
| 1 | 4 | (67%) |

Number of regimens prior to enrollment, n (%)

| | | |
|---|---|---|
| ≥3 | 3 | (50%) |
| Prior immunotherapy, n (%) | 2 | (33%) |

### Clinical Safety Assessments

At the dose of 3 mg/kg, TY21580 has shown a manageable safety and tolerability profile in combination with pembrolizumab, no dose-limiting toxicities was observed. The most frequent TRAEs observed were fatigue (4/6, 67%), pruritus (3/6, 50%) and nausea (3/6, 50%). Two patients had Grade 3 TRAEs: one Grade 3 dehydration (Cycle 3) and one Grade 3 rash (Cycle 1) (Table 4 and Table 5).

TABLE 4
Frequencies of TRAEs with different Grades

| Grade | G1 | G2 | G3 | G4/5 |
|---|---|---|---|---|
| TRAE n (%) | 1 (17%) | 3 (50%) | 2 (33%) | 0 |

TABLE 5
TRAEs at 3 mg/kg TY21580 + Pembrolizumab (200 mg), N = 6

| TRAEs | Grade 1 | Grade 2 | | Grade 3 | Grade 4 |
|---|---|---|---|---|---|
| Fatigue | 2 (33%) | 2 | (33%) | 0 | 0 |
| Pruritus | 0 | 3 | (50%) | 0 | 0 |
| Nausea | 2 (33%) | 1 | (17%) | 0 | 0 |
| Rash | 1 (17%) | 0 | | 1 (17%) | 0 |
| Dehydration | 0 | 0 | | 1 (17%) | 0 |
| Hypertension | 0 | 1 | (17%) | 0 | 0 |
| Vomiting | 0 | 1 | (17%) | 0 | 0 |

| | | | | |
|---|---|---|---|---|
| Constipation | 1 (17%) | 0 | 0 | 0 |
| Chills | 1 (17%) | 0 | 0 | 0 |
| Dyspnoea | 1 (17%) | 0 | 0 | 0 |
| Pyrexia | 1 (17%) | 0 | 0 | 0 |
| Gastritis | 1 (17%) | 0 | 0 | 0 |
| Flatulence | 1 (17%) | 0 | 0 | 0 |
| Hyperuricemia | 1 (17%) | 0 | 0 | 0 |

### Clinical Activity Assessments

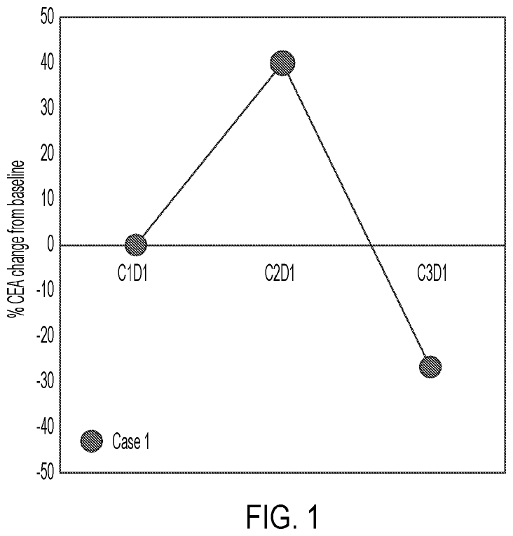

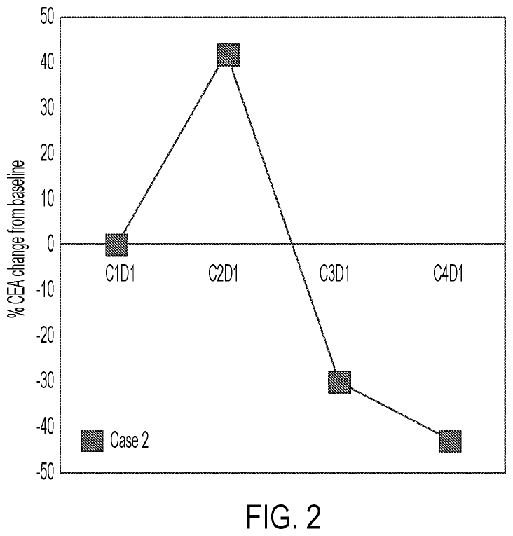

Two MSS CRC patients with lung or liver metastasis showed stable disease (SD) and CEA reduction after the combination treatment. Case 1 is a 47 years old female patient who previously received 5 lines of systemic therapies and had target lesions of pulmonary nodule of 19 and 33 mm at baseline. She showed SD with 4% reduction in sum target lesion and 27% reduction from baseline in CEA levels at the end of cycle 2 (FIG. 1). Case 2 is a 47 year old male patient who previously received 5 lines of systemic therapies and has target lesions in the liver, lymph node and lung of 74, 22 and 20 mm at baseline. He showed stable disease with 3% increase in sum target lesions and 27% reduction from baseline in CEA levels at the end of cycle 2 (FIG. 2).

### Pharmacodynamic Analysis in the Periphery

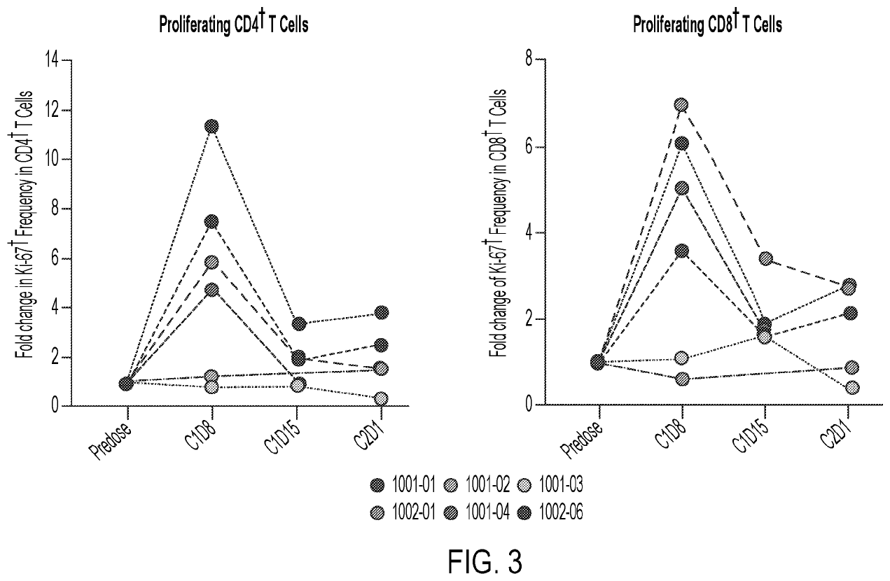

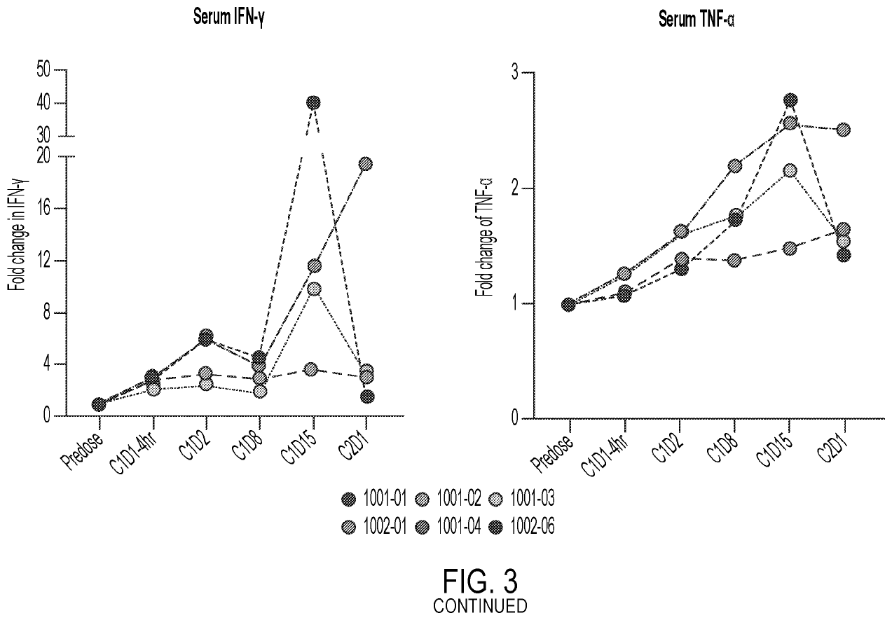

Immune activation was observed in the periphery, treatment by TY21580+pembrolizumab increased peripheral levels of CD4+ and CD8+ T cell proliferation (Ki-67+) shown by flow cytometry analysis and increased serum levels of proinflammatory cytokines including IFN-γ and TNF-α (FIG. 3). All data are compared to that of pre-dose baseline.

### Clinical Pharmacokinetics

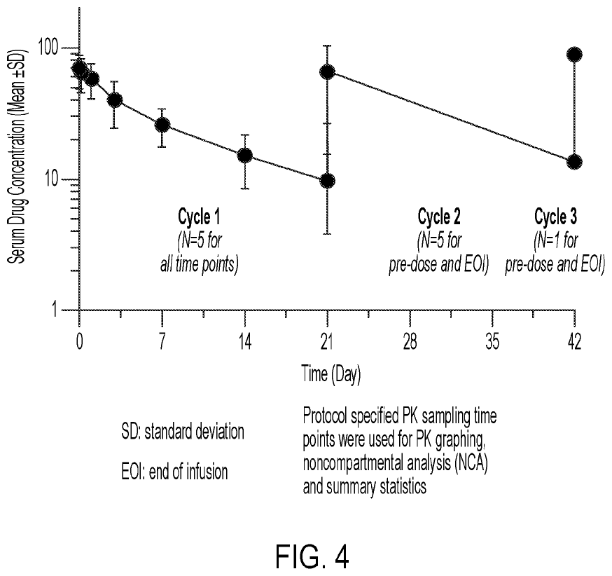

As shown in FIG. 4, combination treatment with pembrolizumab did not alter TY21580 serum PK when compared with TY21580 monotherapy. The mean terminal half-life of TY21580 is estimated to be ˜10 days for Cycle 1 PK, consistent with minimal accumulation after Q3W repeat dosing in this study. Combination treatment has no apparent ADA impact on TY21580 PK.

What is claimed is:

1. A method of treating a cancer in a subject, comprising administering to the subject an effective amount of an anti-CTLA4 antibody and an effective amount of pembrolizumab, wherein the anti-CTLA4 antibody comprises a heavy chain variable region and a light chain variable region, wherein the heavy chain variable region of the antibody comprises an HVR-H1, an HVR-H2, and an HVR-H3, and the light chain variable region of the antibody comprises an HVR-L1, an HVR-L2, and an HVR-L3, wherein the HVR-H1 comprises an amino acid sequence according to a formula YSISSGYHWSWI (SEQ ID NO: 23), the HVR-H2 comprises an amino acid sequence according to a formula LARIDWDDDKYYSTSLKSRL (SEQ ID NO: 35), the HVR-H3 comprises an amino acid sequence according to a formula ARSYVYFDY (SEQ ID NO: 45), the HVR-L1 comprises an amino acid sequence according to a formula RASQSVRGRFLA (SEQ ID NO: 58), the HVR-L2 comprises an amino acid sequence according to a formula DASNRATGI (SEQ ID NO: 66), and the HVR-L3 comprises an amino acid sequence according to a formula YCQQSSSWPPT (SEQ ID NO: 75), wherein the anti-CTLA4 antibody is administered at a dose of from about 3 mg/kg to about 10 mg/kg once every 3 to 6 weeks, and wherein the pembrolizumab is administered at a dose of from about 100 mg to about 300 mg once every three weeks or about 200 mg to about 600 mg every six weeks.

2. The method of claim 1, wherein the anti-CTLA4 antibody is administered at a dose of about 3 mg/kg once every 3 to 6 weeks.

3. The method of claim 1, wherein the anti-CTLA4 antibody is administered at a dose of about 6 mg/kg once every 3 to 6 weeks.

4. The method of claim 1, wherein the pembrolizumab is administered at a dose of about 200 mg once every 3 weeks.

5. The method of claim 1, wherein the pembrolizumab is administered at a dose of about 400 mg once every six weeks.

6. The method of any one of claims 1-5, herein the cancer is resistant or refractory to a prior therapy, wherein the prior therapy is an inhibitor of CTLA4, PD-1, or a PD-1 ligand.

7. The method of claim 6, wherein the prior therapy is ipilimumab.

8. The method of any one of claims 1-7, wherein the cancer is colorectal cancer.

9. The method of claim 8, wherein the CRC is microsatellite stable (MSS) CRC.

10. The method of any one of claims 1-7, wherein the cancer is Kaposi's sarcoma.

11. The method of any one of claims 1-7, wherein the cancer is head and neck squamous cell carcinoma (HNSCC) or angiosarcoma.

12. The method of any one of claims 1-7, wherein the cancer is pancreatic cancer.

13. The method of any one of claims 1-7, wherein the cancer is ovarian cancer.

14. The method of any one of claims 1-13, wherein the cancer is an advanced stage metastatic cancer.

15. The method of claim 14, wherein the cancer has metastasized to the lung or the liver.

16. The method of any one of claims 1-15, wherein the anti-CTLA4 antibody comprises a heavy chain variable region comprising the amino acid sequence of SEQ ID NO: 87 or a variant thereof having at least about 90% sequence identity to the amino acid sequence of SEQ ID NO: 87, and a light chain variable region comprising the amino acid sequence of SEQ ID NO: 100 or a variant thereof having at least about 90% sequence identity to the amino acid sequence of SEQ ID NO: 100.

17. The method of claim 16, wherein the heavy chain variable region comprises the amino acid sequence of SEQ ID NO: 87 and the light chain variable region comprises the amino acid sequence of SEQ ID NO: 100.

18. The method of claim 17, wherein the anti-CTLA4 antibody comprises a heavy chain region comprising the amino acid sequence of SEQ ID NO:126 and the light chain region comprises the amino acid sequence of SEQ ID NO:127.

19. The method of claim 17, wherein the anti-CTLA4 antibody comprises a heavy chain region comprising the amino acid sequence of SEQ ID NO:125 and the light chain region comprises the amino acid sequence of SEQ ID NO:127.

20. The method of any one of claims 1-19, wherein the subject is human.

21. The method of any one of claims 1-20, wherein the anti-CTLA4 antibody and the pembrolizumab are both administered on day 1 of the 3 to 6 week dosing schedule.
